(19) United States
(12) Patent Application Publication
(10) Pub. No.: US 2023/0190718 A1
(43) Pub. Date: Jun. 22, 2023

(54) METHODS FOR THE TREATMENT OF PANCREATITIS AND PREVENTION OF PANCREATIC CANCER

(71) Applicant: BOARD OF REGENTS, THE UNIVERSITY OF TEXAS SYSTEM, Austin, TX (US)

(72) Inventors: Andrea VIALE, Houston, TX (US); Alessandro CARUGO, Houston, TX (US); Edoardo DEL POGGETTO, Houston, TX (US); I-Lin HO, Houston, TX (US)

(73) Assignee: BOARD OF REGENTS, THE UNIVERSITY OF TEXAS SYSTEM, Austin, TX (US)

(21) Appl. No.: 17/998,911

(22) PCT Filed: May 18, 2021
(86) PCT No.: PCT/US2021/033024

§ 371 Date: Nov. 15, 2022

Related U.S. Application Data
(60) Provisional application No. 63/027,209, filed on May 19, 2020.

(51) Int. Cl. A61K 31/437; A61K 31/519; A61K 45/06; A61K 31/56; A61P 1/18

(52) U.S. Cl. CPC A61K 31/437; A61K 31/519; A61K 45/06; A61K 31/56; A61P 1/18

(57) ABSTRACT

Provided herein are methods for the treatment of pancreatitis and/or the prevention of pancreatic cancer. The treatment may comprise the administration of an inducer of acinar-to-ductal metaplasia (ADM), such as an agonist of the mitogen-activated protein kinase (MAPK) signaling pathway, such as a BRAF inhibitor, or an epigenetic modifier, such as a bromodomain extra-terminal motif (BET) inhibitor.

### CROSS-REFERENCE TO RELATED APPLICATIONS

This application claims the benefit of priority of U.S. Provisional Patent Application No. 63/027,209, filed May 19, 2020, incorporated by reference herein in its entirety.

### BACKGROUND

### 1. Field

The present invention relates generally to the field of pharmacology and medicine. More particularly, it concerns compositions and methods for the treatment of pancreatitis and/or prevention of pancreatic cancer.

### 2. Background

The association between tumors and inflammation is a long-established clinical observation (Virchow, 1863). Although many studies demonstrated that the inflammatory microenvironment can promote tumor growth through the activation of survival and proliferation programs in cancer cells (Mantovani et al., 2008; Grivennikov et al., 2010), the reasons why inflammation, an evolutionarily conserved response to damage aimed at reestablishing tissue integrity upon injury, might be integral to tumorigenesis still remain unknown.

PDAC, a tumor characterized by poor prognosis (Ying et al., 2016), represents a distinctive example of cooperation between inflammation and activated oncogenes. Frequently developed in a context of chronic pancreatitis, PDAC is associated with an inflammatory microenvironment (Steele et al., 2013). As supported by a substantial body of evidence across a multitude of experimental models, induction of inflammation in pancreatic tissue expressing oncogenic KRAS hastens tumor progression (Gidekel Friedlander et al., 2009; Guerra et al., 2011), inducing the appearance of neoplastic precursor lesions, such as acinar-to-ductal metaplasia (ADM) and pancreatic intraepithelial neoplasia (PanIN), which can evolve into invasive tumors (Kopp et al., 2012; Liou et al., 2013; Zhang et al., 2013), although alternative models of PanIN-independent progression have been hypothesized (Notta et al., 2016; Real, 2003). Interestingly, preneoplastic pancreatic alterations, specifically ADM, have been previously identified in acute and chronic pancreatitis apparently in the absence of oncogene activation (Storz, 2017; Miyatsuka et al., 2006; Prevot et al., 2012; Sandgren et al., 1990). Because ADM consists in the rapid shut-off of the expression of pancreatic enzymes as a consequence of the acinar cell identity reprogramming, it may represent an adaptive response to inflammation aimed at limiting tissue damage. In this conceptual framework, any genetic and epigenetic events able to promote or stabilize ADM, such as activating mutations of KRAS, may result in the impaired elimination and positive selection of mutant cells within an inflamed tissue. Thus, there is an unmet need to better understand the relationship between inflammatory processes and pancreatic tunorigenesis in order to develop better therapies for the treatment of pancreatitis and prevention of pancreatic cancer.

### SUMMARY

Aspects of the present disclosure are directed methods and compositions for treating pancreatitis. Also disclosed are methods and compositions for preventing pancreatic cancer. The present disclosure includes compositions comprising acinar-to-ductal metaplasia (ADM) inducers, such as MAPK agonists and epigenetic modifiers, and methods of use thereof in treatment of pancreatitis and/or prevention of pancreatic cancer.

Embodiments of the present disclosure include methods for treating pancreatitis, methods for preventing pancreatitis, methods for preventing pancreatic cancer, methods for treating pancreatic cancer, methods for reducing pancreatic inflammation, methods for inhibiting pancreatic tissue damage, methods for pain reduction, methods for inducing ADM, methods for activating MAPK signaling, and compositions comprising ADM inducers. Methods of the disclosure may include at least 1, 2, 3, or more of the following steps: administering an ADM inducer to a subject, administering a MAPK agonist to a subject, administering an epigenetic modifier to a subject, detecting ADM in a subject, diagnosing a subject as having pancreatitis, diagnosing a subject as having pancreatic cancer, administering a cancer therapy to a subject, and administering an anti-inflammatory agent to a subject. Any one or more of the preceding steps may be excluded from certain embodiments of the disclosure. Compositions of the present disclosure may include at least 1, 2, 3, or more of the following components: an ADM inducer, a MAPK agonist, an epigenetic modifier, a cytokine, a BRAF inhibitor, an HDAC inhibitor, a BET inhibitor, and a BRD4 inhibitor. Any one or more of the preceding components may be excluded from certain embodiments of the disclosure.

In one embodiment, the present disclosure provides a method of treating pancreatitis and/or preventing pancreatic cancer in a subject comprising administering an effective amount of an inducer of acinar-to-ductal metaplasia (ADM) to the subject. In particular aspects, the subject is human.

In certain aspects, the method comprises treating or preventing pancreatitis in a subject comprising administering an effective amount of an ADM inducer to the subject. In some aspects, the method comprises treating pancreatitis. In certain aspects, the method comprises preventing pancreatitis. In some aspects, the method comprises preventing pancreatic cancer in a subject comprising administering an effective amount of an ADM inducer to the subject.

In particular aspects, the pancreatic cancer is pancreatic ductal adenocarcinoma (PDAC).

In some aspects, the inducer of ADM is an epigenetic modifier. In specific aspects, the epigenetic modifier is a Bromodomain extra-terminal motif (BET) inhibitor, such as BRD2, BRD3, BRD4, or BRDT inhibitor. In particular aspects, the BET inhibitor is a BRD4 inhibitor. and BRD4 inhibitor. For example, the BRD4 inhibitor is INCB054329, GSK525762A/I-BET762, INCB054329, ABBV-075, OTX015/MK-8628, GSK2820151/I-BET151, PLX51107, ABBV-744, or AZD5153. In some aspects, the epigenetic modifier is a small molecule, peptide, siRNA, sgRNA, proteolysis-targeting chimera (PROTAC) or degron.

In certain aspects, the ADM inducer is a mitogen-activated protein kinase (MAPK) agonist. In some aspects, the MAPK agonist is a BRAF inhibitor, TGFα, or EGF. In particular aspects, the MAPK agonist is TGFα or EGF. In some aspects, the MAPK agonist is a BRAF inhibitor, such as PLX4032 (Vemurafenib), GDC-0879, PLX-4720, sorafenib, dabrafenib (GSK2118436), AZ 628, LGX818, NVP-BHG712. In particular aspects, the BRAF inhibitor is an SOS activator and/or GEF inhibitor. In some aspects, the BRAF inhibitor is PLX4032.

In some aspects, the subject is determined to be RAF wild-type. In certain aspects, the subject is not administered a MEK inhibitor, such as trametinib.

In certain aspects, administering a MAPK agonist prevents KRAS mutations. In particular aspects, administering the ADM inducer prevents or decreases tissue damage and/or inflammation in pancreatic cells as compared to a subject not administered an ADM inducer. In specific aspects, decreased inflammation is measured by decreased inflammatory infiltration, serum inflammatory biochemical markers, edema and pain. In some aspects, decreased tissue damage is measured by serum biochemical markers such as lipase, amylase, trypsinogen and/or lactate dehydrogenase.

In additional aspects, the method further comprises administering at least a second therapy. In some aspects, the at least a second therapy is an anti-inflammatory agent and/or an immunotherapy. In certain aspects, the at least a second therapy is administered concurrently with the ADM inducer. In some aspects, the at least a second therapy is administered sequentially with the ADM inducer. In specific aspects, the at least a second therapy is an anti-inflammatory agent. In some aspects, the anti-inflammatory agent is a non-steroidal anti-inflammatory drug (NSAID) and/or a steroid. In certain aspects, the ADM inducer is administered orally, intraadiposally, intradermally, intramuscularly, intranasally, intraperitoneally, intrarectally, intravenously, liposomally, locally, mucosally, parenterally, rectally, subcutaneously, sublingually, transbuccally, transdermally, via a catheter, via a lavage, via continuous infusion, via infusion, via inhalation, via injection, or via local delivery. In some aspects, the ADM inducer is administered once to the subject. In other aspects, the ADM inducer is administered two or more times to the subject.

A further embodiment provides a composition comprising an effective amount of an ADM inducer for use in the treatment of pancreatitis and/or prevention of pancreatic cancer in a subject.

In some aspects, the ADM inducer is a MAPK agonist. In specific aspects, the MAPK agonist is a BRAF inhibitor, TGFα, or EGF. In particular aspects, the BRAF inhibitor is PLX4032 (Vemurafenib), GDC-0879, PLX-4720, sorafenib, dabrafenib (GSK2118436), AZ 628, LGX818, or NVP-BHG712. In one aspect, the BRAF inhibitor is PLX4032.

In certain aspects, the inducer of ADM is an epigenetic modifier. In some aspects, the epigenetic modifier is a Bromodomain extra-terminal motif (BET) inhibitor. In certain aspects, the BET inhibitor is a BRD4 inhibitor, such as INCB054329, GSK525762A/I-BET762, INCB054329, ABBV-075, OTX015/MK-8628, GSK2820151/I-BET151, PLX51107, ABBV-744, or AZD5153. In certain aspects, the epigenetic modifier is a small molecule, peptide, siRNA, sgRNA, PROTAC or degron.

In particular aspects, the subject is human. In some aspects, the pancreatitis is chronic pancreatitis or acute pancreatitis. In particular aspects, the pancreatic cancer is PDAC.

In some aspects, the ADM inducer prevents KRAS mutations, tissue damage, and/or inflammation.

In additional aspects, the method further comprises at least a second therapy. In some aspects, the at least a second therapy is an anti-inflammatory agent and/or immunotherapy. In certain aspects, the at least a second therapy is an anti-inflammatory agent. In particular aspects, the anti-inflammatory agent is a steroid and/or an NSAID.

Another embodiment provides a method of inhibiting pancreatic tissue damage and/or inflammation in a subject comprising administering an effective amount of an ADM inducer to the subject.

In some aspects, the inducer of ADM is an epigenetic modifier. In specific aspects, the epigenetic modifier is a Bromodomain extra-terminal motif (BET) inhibitor, such as BRD2, BRD3, BRD4, or BRDT inhibitor. In particular aspects, the BET inhibitor is BRD4 inhibitor. For example, the BRD4 inhibitor is INCB054329, GSK525762A/I-BET762, INCB054329, ABBV-075, OTX015/MK-8628, GSK2820151/I-BET151, PLX51107, ABBV-744, or AZD5153. In some aspects, the epigenetic modifier is a small molecule, peptide, siRNA, sgRNA, PROTAC or degron.

In certain aspects, the ADM inducer is a mitogen-activated protein kinase (MAPK) agonist. In some aspects, the MAPK agonist is a BRAF inhibitor, TGFα, or EGF. In particular aspects, the MAPK agonist is TGFα or EGF. In some aspects, the MAPK agonist is a BRAF inhibitor, such as PLX4032 (Vemurafenib), GDC-0879, PLX-4720, sorafenib, dabrafenib (GSK2118436), AZ 628, LGX818, NVP-BHG712. In particular aspects, the BRAF inhibitor is an SOS activator and/or GEF inhibitor. In some aspects, the BRAF inhibitor is PLX4032.

As used herein the specification, “a” or “an” may mean one or more. As used herein in the claim(s), when used in conjunction with the word “comprising,” the words “a” or “an” may mean one or more than one.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that embodiments described herein in the context of the term “comprising” may also be implemented in the context of the term “consisting of” or “consisting essentially of.”

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

The term “essentially” is to be understood that methods or compositions include only the specified steps or materials and those that do not materially affect the basic and novel characteristics of those methods and compositions.

As used herein, a composition or media that is “substantially free” of a specified substance or material contains≤30%, ≤20%, ≤15%, more preferably ≤10%, even more preferably ≤5%, or most preferably 1% of the substance or material.

The terms “substantially” or “approximately” as used herein may be applied to modify any quantitative comparison, value, measurement, or other representation that could permissibly vary without resulting in a change in the basic function to which it is related.

Throughout this application, the term “about” is used to indicate that a value includes the inherent variation of error for the measurement or quantitation method.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

A variety of embodiments are discussed throughout this application. Any embodiment discussed with respect to one aspect applies to other aspects as well and vice versa. Each embodiment described herein is understood to be embodiments that are applicable to all aspects. It is contemplated that any embodiment discussed herein can be implemented with respect to any method or composition, and vice versa. Furthermore, compositions and kits can be used to achieve methods disclosed herein.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawings(s) will be provided by the Office upon request and payment of the necessary fee.

The following drawings form part of the present specification and are included to further demonstrate certain aspects of the present invention. The invention may be better understood by reference to one or more of these drawings in combination with the detailed description of specific embodiments presented herein.

FIGS. 1A-1H: Transient inflammation promotes tumor progression long after resolution. FIG. 1A. Schematics representing the experimental design. Briefly iKRAS mice are treated for two days (−D2-D1) with caerulein (CAE) to induce acute pancreatitis then monitored for 4 weeks. When pancreata are fully recovered from pancreatitis (D28), CAE-treated and control mice are put on doxycycline to induce the expression of mutated KRAS and followed for tumor development. FIG. 1B. Histological analysis of pancreatic samples at different time points after pancreatitis induction. Edema and infiltration appear at the end of CAE treatment, increase at day 1 (D1) and are completely resolved by day 7 (D7) when pancreata reacquire the normal structure (scale bar-100 μm). FIG. 1C. Immunostaining for CD45 and Ki67 of pancreatic samples at different time points after pancreatitis induction. Strong intra-lobular infiltration of CD45 positive cells is present at day 1 (D1) after CAE treatment and signal disappears by day 7 (D7). Similarly, Ki67 staining is strongly increased at day 1 (D1) when many different cells show positivity, then signal decreases over time and disappears by day 28 (D28) (scale bar-100 μm). FIG. 1D. Immunofluorescence for NFkB (p-Ser536, red), ductal marker DBA (green) and DAPI (blue) of pancreatic samples at different time points. Only at day 1 (D1) nuclei of different cell types, mainly epithelial, are positive for NFkB, suggesting that inflammation is molecularly resolved at later time points (scale bar-50 μm). FIG. 1E. Kaplan-Meier survival of mice previously exposed to inflammation (caerulein, n=21) or control mice (untreated, n=10) after KRAS induction. FIG. 1F. MRI scan of two animals, tumor (T), stomach (S), bowel (B) and kidney (K) are indicated. FIG. 1G. Histology of a tumor derived from an animal previously exposed to caerulein (scale bar-100 μm). FIG. 1H. Immunostaining for cytokeratin-19 (KRT19) and amylase (AMY2A) of the same tumor as in g (scale bar-100 μm).

FIGS. 2A-2G: Cell autonomous effects of resolved inflammation. FIG. 2A. Relative organogenic potential of cells sorted from single cell suspension of pancreata isolated from Dclk1-DTR-ZsGreen mice based on their fluorescence: ZsGreen positive (Dclk1+), ZsGreen negative (Dclk1−) (n=3). FIG. 2B. Green organoids derived from p48Cre-mT/mG mice are orthotopically transplanted in pancreata of animals 48 hours after CAE treatment. Cryosections of pancreata from mice sacrificed at 4-week after implantation revealed GFP-positive lobuli. GFP (green), DAPI (blue). FIG. 2C. Schematics representing the experimental design. Briefly, organoids derived from iKRAS pancreas recovered from pancreatitis (4-weeks recovery) and control animals are orthotopically injected in recipient animals never exposed to inflammation. Then KRAS expression is induced and mice followed for tumor development. FIG. 2D. Quantification of organoid size evaluated as pixel log 10 scale (9 fields for each condition; CAE n=69, CTRL n=58; p<0.01) and representative picture of organoids derived from pancreata of mice recovered from inflammation (CAE) or controls (CTRL). FIG. 2E. Kaplan-Meier survival of mice transplanted with iKRAS organoids derived from pancreas recovered from pancreatitis (caerulein, n=7) and control mice (untreated, n=5) after KRAS induction. FIG. 2F. Histology of orthotopic tumors developed from animals injected with organoids derived from recovered inflammation and corresponding liver metastasis, left panels. Immunostaining for GFP and CD45 of the primary and secondary lesions, middle and right panels (scale bar-100 m). FIG. 2G. Immunofluorescence for Dclk1 (Red), CD45 (Green) and DAPI (Blue) of an orthotopic tumor developed from animals injected with organoids derived from recovered inflammation (scale bar-50 m). Data are mean±standard deviation.

FIGS. 3A-3F: Pervasive transcriptional deregulation in epithelial cells recovered from inflammation. FIG. 3A. Heat map showing normalized expression values of 857 differentially expressed genes after treatment with CAE. Blue and orange colors indicate down- and up-regulated genes, respectively. FIG. 3B. GSEA enrichment plots showing the hallmark signature Kras signaling and Development and Progression signature including genes coregulated during development and carcinogenesis in pancreatic cells (19). The p53 Pathway signature, which is enriched in down-regulated genes is also shown. Genes are ranked from left to right based on signed p-value, with genes on the left showing significantly higher expression after CAE treatment. NES, Normalized enrichment score; FDR, false discovery rate. FIG. 3C. IPA analysis of pathways associated with diseases or biological functions (Diseases and Bio Functions). Highest-ranked terms are shown. FIG. 3D. Scatter plots showing differential H3K27Ac enrichment at genomic regions in CAE treated vs. control animals. Hypo- and Hyper acetylated regions are represented as blue and red dots, respectively. All other acetylated regions are represented as grey dots. FIG. 3E. TF binding sites over-representation at promoters and distal regions. The over-represented families of TFs in the promoters of up-regulated (Up-P) and down-regulated (Down-P) genes relative to all Refseq genes are shown on the left. The right panel shows the over-represented TF families in the differentially acetylated TSS-distal regions (using the FANTOM5 collection of enhancers as background). The heat map shows the negative logarithm of the enrichment P-value determined by a two-tailed Welch's t-test. FIG. 3F. Immunofluorescence for ductal marker DBA (Green), DAPI (blue) and Egr1 (Red, upper panels) or Sox9 (Red, lower panels) at different time points (day1 D1, day 28 D28) after induction of inflammation in wild type animals (scale bar-20 μm). Quantification of nuclear signal as pixel log 10 intensity for EGR1 (top right) and SOX9 (bottom right). An average of 3,800 nuclei from at least seven 40× fields of pancreatic tissue from 3 to 5 mice each experimental group were counted and used for the analysis.

FIGS. 4A-4G: 116 is a mediator of epithelial memory. FIG. 4A. Schematics representing the experimental design. Briefly, organoids derived from iKRAS mice are co-cultured in presence or absence of CD45 positive cells isolated from an acute pancreatitis. After one week, conditioned organoids are moved to conventional medium for other 4 weeks and then transplanted orthotopically in recipient mice and KRAS induced. FIG. 4B. Kaplan-Meier survival of mice transplanted with conditioned (CD45, n=5) or not conditioned organoids (CTRL, n=7) after KRAS induction. FIG. 4C. Cytokine array of medium conditioned for 1 or 7 days with CD45, absorbance for different antibodies is reported. FIG. 4D. Immunoblotting for pStat3 (phosphor Tyr 705), Stat3 and Vinculin of organoids exposed to CD45 conditioned medium (top panel) or Hyper-IL6 200 ng/ml (bottom panel) for indicated time points. FIG. 4E. Immunofluorescence for IL6 (red), pSTAT3 (green) and DAPI (blue) of pancreatic sample at day 1 after caerulein treatment showing a multitude of pSTAT3 nuclear positive cells, including many acinar structures (yellow dashed lines), interspersed among IL6 positive cells (scale bar-50 μm). FIG. 4F. CyTOF immunophenotyping of CD45 positive cells infiltrating the pancreas during acute pancreatitis, tSNE-plots for CD68, CD11, F4/80 and IL6 are reported. FIG. 4G. Immunoblotting for pStat3 (phosphor Tyr 705), Stat3, Egr1, Runx1, Ets1, Sox9 and Vinculin of organoids exposed to Hyper-IL6 200 ng/ml for 24 hours and then sampled at indicated time points after Hyper-IL6 wash-out. Data are mean±standard deviation.

FIGS. 5A-5H: ADM as a physiological and reversible adaptation to limit tissue damage. FIG. 5A. Schematics representing the experimental design. To investigate the role of epithelial memory, wild type or iKRAS mice were rechallenged with a second acute pancreatitis after the complete recovery from a previous one. Pharmacologic modulation of ADM or KRAS induction was obtained by treating mice with EGF, MEK inhibitor or doxycycline (KRAS induction) the day before the second administration of caerulein. FIG. 5B. Levels of amylase detected in the peripheral blood at 24 hs after the induction of acute pancreatitis (−D1) in WT mice; untreated mice (CTRL, n=2), mice without memory after a single inflammation (Single, n=2), mice with memory after rechallenging (Rechallenged, n=2). FIG. 5C. Levels of LDH detected in the peripheral blood at 24 hs after the induction of acute pancreatitis (−D1) in WT mice; untreated mice (CTRL, n=2), mice without memory after a single inflammation (Single, n=2), mice with memory after rechallenging (Rechallenged, n=2). FIG. 5D. Histology of pancreata of WT mice at 24 hs after the induction of acute pancreatitis (−D1) with (Rechallenged) or without memory (Single Inflammation) (left panels, scale bar-50 μm); Immunofluorescence for cleaved caspase 3 (CC3-Red), and DAPI (Blue) same setting as before (right panels, scale bar-50 μm). Green channel (BG), although unstained, was acquired and used to highlight tissue architecture and vessel. FIG. 5E. Immunofluorescence for cytokeratin-19 (KRT19-Red), amylase (AMY2A-Green) and DAPI (Blue) at 24 hs (Day 1) after a 2 day inflammation in wild type mice with (Rechallenged) or without memory (Single Inflammation) (scale bar-50 μm). FIG. 5F. Upper panels: histology of pancreata of iKRAS mice at 24 hs (Day 1) after rechallenging in presence/absence of pharmacological treatment with EGF, MEK inhibitor or induction of KRAS (scale bar-100 μm). Lower panels: Damage evaluation in iKRAS-rechallenged mice, immunofluorescence for cleaved caspase 3 (CC3-Red) and DAPI (Blue) at 24 hs (Day 1) after rechallenging in presence/absence of pharmacological treatment with EGF, MEK inhibitor or induction of KRAS. Green channel (BG), although unstained, has been acquired and used to highlight tissue architecture and vessel (scale bar-100 μm). FIG. 5G. ADM relative area quantification, same setting as in f upper panels; rechallenging (CTRL, n=4), rechallenging plus EGF (EGF, n=4), rechallenging plus MEK inhibitor (MEKi, n=4), rechallenging plus KRAS induction (KRAS, n=4). FIG. 5H. Pancreatic damage quantification evaluated as cleaved caspase 3 positive area (Log 10 scale), same setting as in f lower panels; rechallenging (CTRL, n=8), rechallenging plus EGF (EGF, n=9), rechallenging plus MEK inhibitor (MEKi, n=7), rechallenging plus KRAS induction (KRAS, n=6). Data are mean±standard deviation.

FIGS. 6A-6D: FIG. 6A. Immunostaining for Ki67 of pancreatic samples at day 1 (D1) after CAE treatment showing the different nature of Ki67-positive cells: interacinar stroma (1), acinar (2), centroacinar (3) (scale bar-100 μm). FIG. 6B. Immunofluorescence for Ki67 (White), DBA (Green) and DAPI (Red) of pancreatic samples at day 1 (D1) after CAE treatment or control pancreas (CTRL) showing the different nature of Ki67-positive cells: ductal (4), acinar (2) (scale bar-20 μm). FIG. 6C. Immunofluorescence for Ki67 (White), DBA (Green) and CD45 (Red) of pancreatic samples at day 1 (D1) after CAE treatment showing activated CD45 positive cells infiltrating the tissue (scale bar-100 μm). FIG. 6D. Immunofluorescence for pSTAT3 (Green) and DAPI (Blue) of pancreatic samples at different time points after inflammation induction. Only at day 1 (D1) cells show strong nuclear signals (scale bar-50 μm). FIG. 6E. Kaplan-Meier survival of mice same as in FIG. 1E also showing mice in which KRAS expression was induced before induction of inflammation (Conventional, n=7).

FIGS. 7A-7G: FIG. 7A. Construct for the generation of the Dclk1-DTR-ZsGreen mouse model. FIG. 7B. Density plots representing sorting gates for pancreatic cells isolated from Dclk1-DTR-ZsGreen animals. FIG. 7C. Quantification of organoids number per 105 cells plated from pancreas of wild type mice recovered from inflammation (CAE, n=3) or controls (CTRL, n=3). FIG. 7D. Immunofluorescence for cadherin E (CDH1, Red), cytokeratin 19 (KRT19, Green) and DAPI (Blue) of organoids derived from control or CAE recovered animals (confocal microscopy). FIG. 7E. 3D reconstruction of organoids in confocal microscopy stained with DBA (Green) and DAPI (Blue), corresponding size is reported (right panels). FIG. 7F. Immunostaining for cytokeratin 19 (KRT19) and amylase (AMY2A) of orthotopic tumors from animals injected with organoids derived from recovered inflammation and corresponding liver metastasis (scale bar-100 μm). FIG. 7G. Immunostaining for Dclk1 of orthotopic tumor from animals injected with organoids derived from recovered inflammation (scale bar-100 μm). Data are mean±standard deviation.

FIGS. 8A-8D: FIG. 8A. Heat map showing normalized expression values of 59 differentially expressed TFs. Blue and orange colors indicate down- and up-regulated genes, respectively. FIG. 8B. Immunostaining for SOX9 (Red), ductal marker DBA (Green), DAPI (blue) of pancreas at day 28 (D28) after induction of inflammation in wild type mice (scale bar-20 μm). FIGS. 8C-8D. Immunostaining for RUNX1 (FIG. 8C) and ETS1 (FIG. 8D) (Red), DAPI (Blue) at different time points (day1 D1, day 28 D28) after induction of inflammation in wild type mice. Green channel (BG), although unstained, has been acquired and used to highlight tissue architecture (scale bar-20 μm). Quantification of nuclear signal as pixel log 10 intensity for RUNX1 (FIG. 8C) and ETS1 (FIG. 8D) (lower panels). An average of 3,800 nuclei from at least seven 40× fields of pancreatic tissue from 3 to 5 mice each experimental group were counted and used for the analysis.

FIG. 9: Immunofluorescence for EGR1, SOX9, RUNX1 and ETS1 (Red) and DAPI (Blue) on human samples of chronic pancreatic inflammation. Green channel (BG), although unstained, has been acquired and used to highlight tissue architecture (scale bar-50 μm).

FIGS. 10A-10D: FIGS. 10A-10B. Histology and immunostaining for GFP of tumors developed from animals injected with CD45 conditioned organoids (scale bar-100 μm). FIG. 10C. Picture of the cytokine array used to quantify cytokines present in medium after conditioning with CD45-positive cells. FIG. 10D. CyTOF immunophenotyping of CD45 positive cells infiltrating the pancreas during acute pancreatitis, tSNE-plots for CD4, CD8, B220 and NK1.1 are reported.

FIGS. 11A-11F: FIG. 11A. Histology of wild type pancreata with or without memory (Rechallenged or Single Inflammation respectively) at 24 hs after induction of acute pancreatitis (−Day 1) (scale bar-100 μm). FIG. 11B. Immunofluorescence for cleaved caspase 3 (CC3-Red) and DAPI (Blue) of wild type pancreata with or without memory (Rechallenged or Single Inflammation, respectively) at 24 hs after induction of acute pancreatitis (scale bar-50 μm). Green channel (BG), although unstained, has been acquired and used to highlight tissue architecture and vessel. FIG. 11C. Histology of wild type pancreata with or without memory (Rechallenged or Single Inflammation, respectively) before and after 2-day caerulein treatment (Day 1 and Day 7) (scale bar-100 μm). FIG. 11D. Immunofluorescence for cytokeratin-19 (KRT19, Green), amylase (AMY2A, Red) and DAPI (Blue) of wild type pancreata with or without memory (Rechallenged or Single Inflammation, respectively) before and after 2-day caerulein treatment (Day 1 and Day 7) (scale bar-50 μm). FIG. 11E. Detail of immunostaining for cytokeratin-19 (KRT19-Green), amylase (AMY2A-Red) and DAPI (Blue) of wild type pancreata at Day 1 after caerulein treatment in rechallenged mice (60× magnification). FIG. 11F. Representative histology of iKRAS pancreata at 28 days from resolved inflammation after pharmacological treatment with EGF or MEK inhibitor (scale bar-50 μm).

FIGS. 12A-12B: FIG. 12A. Inflammatory infiltration evaluated with immunohistochemistry for CD45 at 24 hrs after caerulein treatment in presence/absence of pharmacological treatment with Sulindac or EGF. Two different low magnification fields and one high magnification field for each treatment are shown. Red asterisks highlight lymphoid tissue as an internal positive control for the staining. FIG. 12B. CD45+ cells quantification, counts of CD45+ cells per field normalized to control (CTRL). Average+/−SD (n=5).

FIGS. 13A-13B: FIG. 13A. Evaluation of EGF or Vemurafenib treatment in a context of Caerulein-induced pancreatitis. Upper panel: representative histology of pancreata 24 hs after Caerulein administration in presence/absence of pharmacological treatment with EGF or Vemurafenib. Middle panel: Immunofluorescence for p-ERK of pancreata 24 hs after Caerulein administration in presence/absence of pharmacological treatment with EGF or Vemurafenib. Lower panel: Immunofluorescence for CD45 of pancreata 24 hs after Caerulein administration in presence/absence of pharmacological treatment with EGF or Vemurafenib. FIG. 13B. CD45+ cells quantification, counts of CD45+ cells per field normalized to control (CTRL). Average+/−SD (n=5).

FIGS. 14A-14B: FIG. 14A. Evaluation of EGF or Brd4 inhibitor (INCB054329) treatment in a context of Caerulein-induced pancreatitis. Upper panel: representative histology of pancreata 24 hs after Caerulein administration in presence/absence of pharmacological treatment with EGF or Brd4 inhibitor. Lower panel: Inflammatory infiltration evaluated with immunohistochemistry for CD45 at 24 hrs after Caerulein treatment in presence/absence of pharmacological treatment with EGF or Brd4 inhibitor. FIG. 14B. CD45+ cells quantification, counts of CD45+ cells per field normalized to control (CTRL). Average+/−SD (n=5).

### DETAILED DESCRIPTION

The present studies investigated the long-term effects of inflammatory events in response to acute pancreatic damage, and how resolved inflammation cooperates with activated oncogenes to drive tumor progression in normal epithelial cells. The present disclosure is based, at least in part, on the surprising discovery that acinar-to-ductal metaplasia (ADM) protects against pancreatic tissue damage and that induction of ADM (e.g., via administration of an ADM inducer such as a MAPK agonist or epigenetic modifier) is effective in reducing pancreatic inflammation.

Inflammation is one of the major risk factors for pancreatic ductal adenocarcinoma (PDAC). When occurring in the context of pancreatitis, mutations of KRAS, the most frequent driver oncogene of pancreatic cancer, lead to accelerated tumor development through the sequential occurrence of ADM, dysplastic lesions, and eventually overt PDAC. The present studies demonstrated that since activating mutations of KRAS maintain an irreversible ADM and thus limit cellular and tissue damage, they are beneficial and under strong positive selection in the context of recurrent pancreatitis. To demonstrate that ADM is a physiologic, fast and reversible adaptation that limits the detrimental effects of repeated pancreatitis, the effects of pharmacological modulation of ADM were evaluated.

To this purpose different pharmacological modalities and classes of molecules were used to induce ADM. First, since ADM is mediated through activation of MAPK signaling, EGF was used as a surrogate for a MAPK activator. The present studies demonstrated that administration of EGF at pharmacological doses before induction of pancreatitis largely promoted ADM formation. In EGF treated animals, there was a significant decrease of CD45 infiltration, a well-known marker of inflammation, compared to the current standard of care, such as NSAID (e.g. Sulindac) (FIG. 12A-B). Importantly, these findings were accompanied by strong reduction of tissue damage, evaluated as the number of cells positive for cleaved caspase 3.

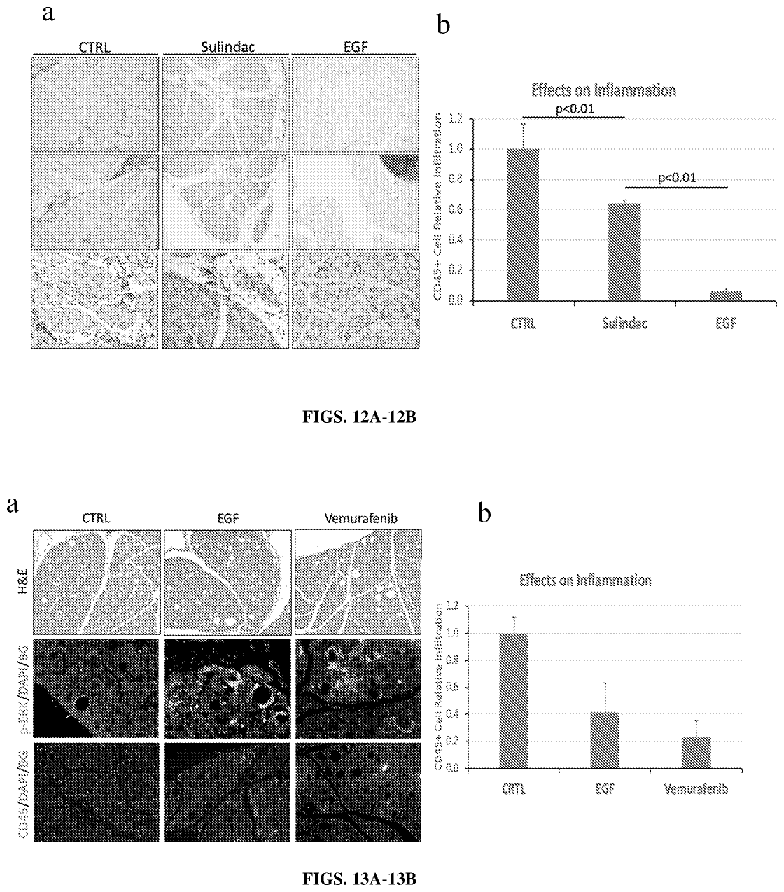

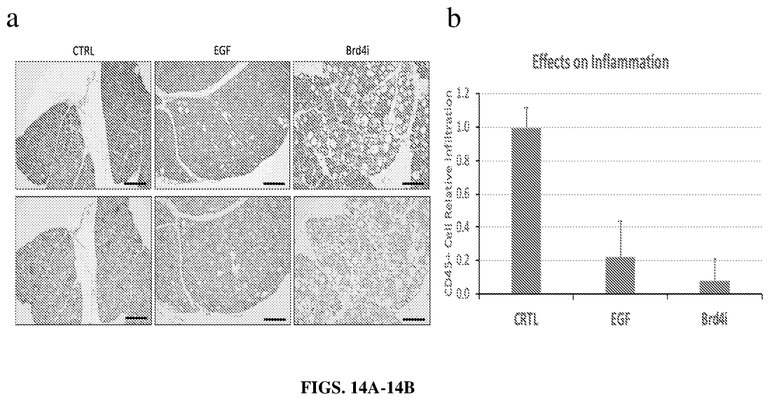

In another strategy to induce ADM, a RAF inhibitor, such as PLX4032 (Vemurafenib), was used which is known to activate MAPK signaling in BRAF wildtype cells. The small molecule inhibitor when administered before the development of pancreatitis was able to induce ADM further limiting inflammation when compared to EGF (FIG. 13A-B).

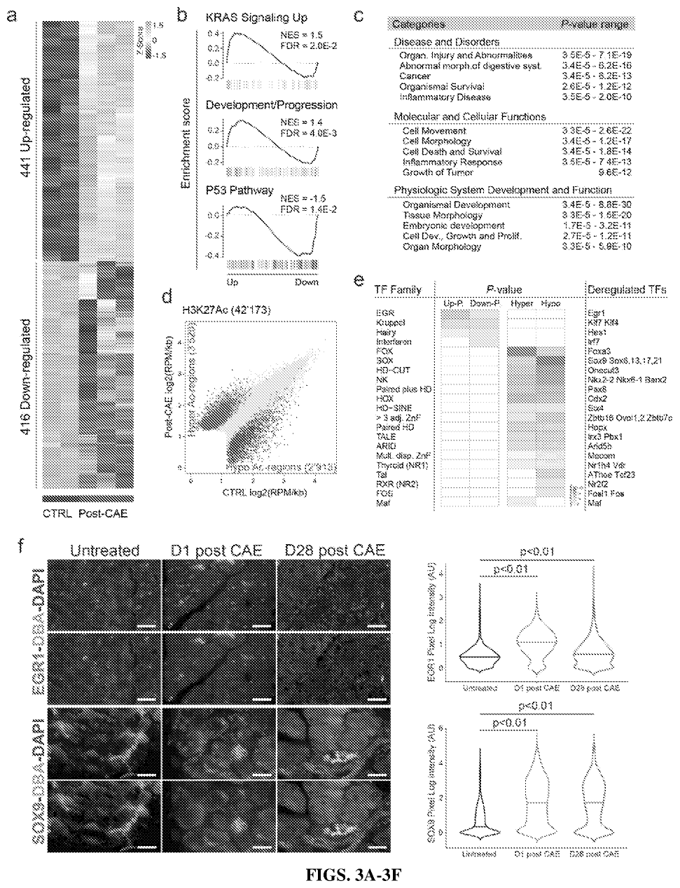

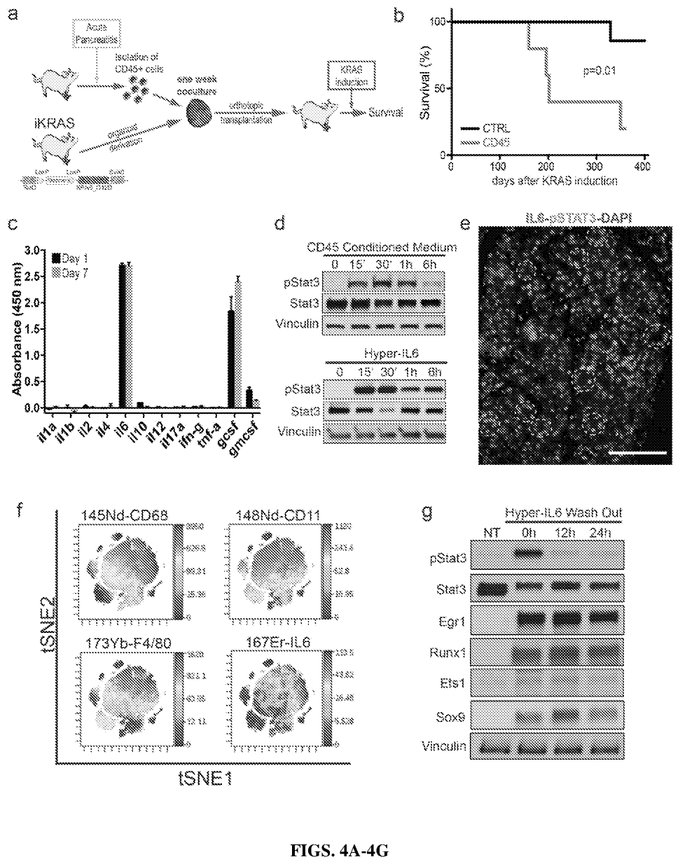

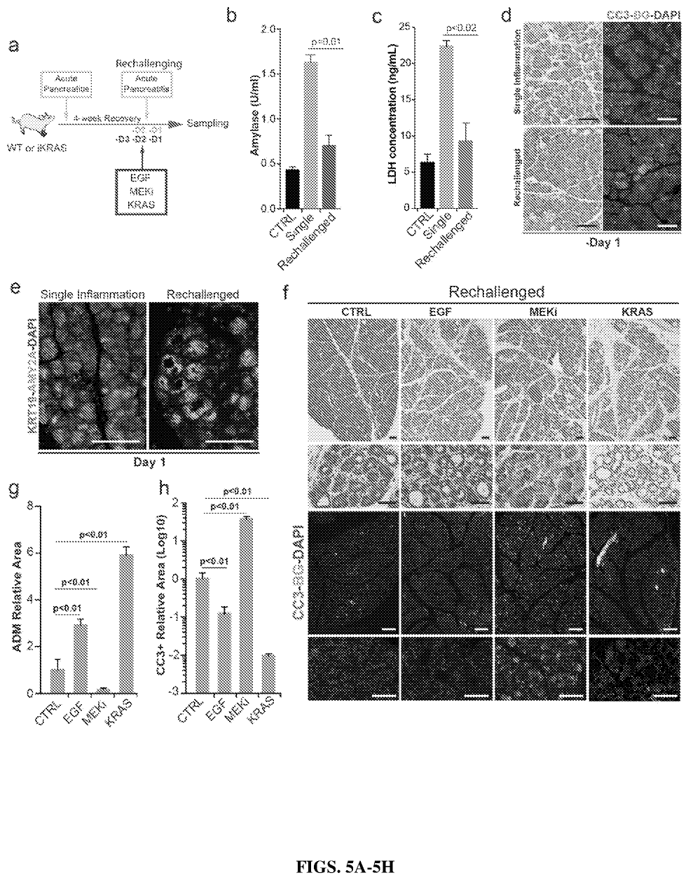

Next, an epigenetic approach was used to induce ADM. As the disclosed studies showed that epithelial memory is mediated by extensive and persistent chromatin modifications such as histone acetylation (e.g., H3K27Ac) mainly in regions located distally from gene promoters (e.g., enhancers) (FIG. 3D-E), bromodomain extra-terminal motif (BET) inhibitors were tested as ADM inducers. As shown in FIG. 14A-B, administration of a Brd4 inhibitor (e.g., INCB054329) led to significant ADM induction and a strong suppression of inflammation evaluated by measurement of CD45 infiltration upon pharmacological induction of pancreatitis (FIG. 14A-B). Taken together, these data support a model in which an agent able to induce ADM, such as MAPK agonists and/or chromatin modifiers interfering with the gene program responsible for the maintenance of the acinar identity, can be used to minimize tissue damage by blocking the production of acinar zymogens during inflammatory events and at the same time alleviate the strong selective pressure to mutate KRAS preventing the development of pancreatic cancer.

Accordingly, in certain embodiments, the present disclosure provides methods for the treatment of pancreatitis and/or the prevention of pancreatic cancer development. A subject with pancreatitis may be administered an ADM inducer, such as a MAPK agonist (e.g., TGFα, EGF, or any pharmacological compound able to activate MAPKs, such as a RAF inhibitor) as well as epigenetic drugs able to perturb the transcriptional programs involved in the maintenance of acinar cell identity, such as inhibitors of the Bromodomain and Extra-terminal (BET) proteins (e.g., BRD4 inhibitors). As demonstrated in the present studies, any of these ADM inducers may be used to ameliorate pancreatitis by protecting pancreatic cells from tissue damage while also reducing the positive pressure to mutate KRAS and, eventually, the progression to PDAC.

The current therapeutic options for patients diagnosed with pancreatitis are symptomatic and based on anti-inflammatory agents (e.g., steroid and/or non-steroidal anti-inflammatory drugs, NSAIDs) and support treatments. The present approach, which can quickly reduce the enzymatic content of acinar cells through the induction of reversible acinar-to-ductal metaplasia (ADM), is curative by preventing and limiting the pancreatic damage derived from further release of pancreatic enzymes along with preserving organ functionality.

### I. ADM INDUCERS

In certain embodiments, the present disclosure provides ADM inducers for the treatment or prevention of pancreatitis and/or pancreatic cancer. The term “ADM inducer” (also “inducer of ADM”) as used herein refers to any agent that suppresses the gene program responsible for the maintenance of the acinar identity and induces reversible acinar to ductal metaplasia (ADM). Examples of ADM inducers are provided below and elsewhere herein.

A. MAPK Agonists

In some embodiments, the ADM inducer is a MAPK agonist. A mitogen-activated protein kinase (MAPK) is a type of protein kinase that is specific to the amino acids serine and threonine (i.e., a serine/threonine-specific protein kinase). MAPKs are involved in directing cellular responses to a diverse array of stimuli, such as mitogens, osmotic stress, heat shock and proinflammatory cytokines. They regulate cell functions including proliferation, gene expression, differentiation, mitosis, cell survival, and apoptosis.

The term “MAPK signaling pathway” is used to describe the downstream signaling events attributed to Mitogen-activated protein (MAP) kinases. The mitogen-activated protein kinase (MAP kinase) pathways consist of four major groupings and numerous related proteins which constitute interrelated signal transduction cascades activated by stimuli such as growth factors, stress, cytokines and inflammation. Signals from cell surface receptors such as GPCRs and growth factor receptors (e.g., receptor tyrosine kinases or RTKs) are transduced, directly or via small G proteins such as Ras and Rac, to multiple tiers of protein kinases that amplify these signals and/or regulate each other. Mitogen-activated protein (MAP) kinases are important players in signal transduction pathways activated by a range of stimuli and mediate a number of physiological and pathological changes in cell function. There are three major subgroups in the MAPK family: ERK, p38, and JNK/SAPK. ERK is activated mainly by mitogenic stimuli, whereas p38 and JNK/SAPK are activated mainly by stress stimuli or inflammatory cytokines MAP kinases are part of a three-tiered phosphorylation cascade and MAP kinase phosphorylation on a threonine and tyrosine residue located within the activation loop of kinase subdomain VIII results in activation. However, this process is reversible even in the continued presence of activating stimuli, indicating that protein phosphatases provide an important mechanism for MAP kinase control. Dual specificity phosphatases (DSP's) from tyrosine phosphatase (PTP) gene superfamily are selective for dephosphorylating the critical phosphothreonine and phosphotyrosine residues within MAP kinases. Ten members of dual specificity phosphatases specifically acting on MAPKs, termed MAPK phosphatases (MKPs), have been reported. They share sequence homology and are highly specific for MAPK's but differ in the substrate specificity, tissue distribution, subcellular localization, and inducibility by extracellular stimuli. MKPs have been shown to play important roles in regulating the function of the MAPK family. DSP gene expression is induced strongly by various growth factors and/or cellular stresses. Expression of some gene family members, including CLlOO/MKP-1, hVH-2/MKP-2, and PACl, is dependent at least in part on MAP kinase activation providing negative feedback for the inducing MAP kinase or for regulatory cross talk between parallel MAP kinase pathways. DSPs are localized to different subcellular compartments and certain family members appear highly selective for inactivating distinct MAP kinase isoforms. This enzymatic specificity is due to catalytic activation of the DSP phosphatase after tight binding of its amino-terminal to the target MAP kinase. Thus, DSP phosphatases provide a sophisticated mechanism for targeted inactivation of selected MAP kinase activities. p38 MAPKs are members of the MAPK family that are activated by a variety of environmental stresses and inflammatory cytokines. Stress signals are delivered to this cascade by members of small GTPases of the Rho family (Rac, Rho, Cdc42). As with other MAPK cascades, MAPKKK, typically a MEKK or a mixed lineage kinase (MLK), phosphorylates and activates MKK3/5, the p38 MAPK kinase. MKK3/6 can also be activated directly by ASKI, which is stimulated by apoptotic stimuli. P38 MAK is involved in regulation of Hsp27 and MAPKAP-2 and several transcription factors including ATF2, STATl, the Max/Myc complex, MEF-2, ELK-I and indirectly CREB via activation of MSKl.

In certain embodiments, the present disclosure concerns MAPK agonist compounds. The term “MAPK agonist” as used herein refers to any agent which increases, enhances, or positively modulates the activation of MAPKs and/or their upstream and/or downstream signaling pathways. An agent can be a drug, a small molecule, such as a chemical entity, a peptide, a protein, a growth factor (including e.g., TGFα, EGF), a chimeric molecule, an antibody, antibody fragment or other such agent, etc. An agent which is an agonist of MAPK may include a kinase inhibitor, phosphatase, etc. In certain embodiments, the agonist can be a small molecule, peptide, siRNA, sgRNA, PROTAC or degron. For example, the CRISPR gene-editing system may be used to activate the MAPK pathway.

The term an “agonist” refers to an agent that binds to a polypeptide or polynucleotide and stimulates, increases, activates, facilitates, enhances activation, sensitizes or up regulates the activity or expression of the polypeptide or polynucleotide. An agonist may inhibit or activate signaling pathways according to its action. An agonist can also be termed an “activator” which is an agent that, e.g., induces or activates the expression of a polypeptide or polynucleotide or binds to, stimulates, modulates, increases, opens, activates, facilitates, enhances activation, DNA binding or enzymatic activity, sensitizes or upregulates the activity of a polypeptide or polynucleotide, e.g., agonists. Activation is achieved when the activity value of a polypeptide or polynucleotide is significantly higher relative to the control, for example at least 110%, 150%, 200-500%, or 1000-3000% higher, or any range or value derivable therein.

Exemplary MAPK agonists for use in the present methods include, but are not limited to, TGFα, EGF, or any pharmacological compound able to activate MAPKs or positively modulate MAPK signaling. For example, the MAPK agonist may be a RAF inhibitor, where such a RAF inhibitor positively modulates MAPK signaling, such as PLX4032 (Vemurafenib), sorafenib (e.g., sorafenib tosylate), PLX-4720, dabrafenib (GSK2118436), GDC-0879, AZ 628, LGX818, and NVP-BHG712, as well as any positive modulator/enhancer of RAS activity (e.g., Son of Sevenless (SOS) activators and/or guanine nucleotide exchange factor (GEF) inhibitors). In some embodiments, the RAF inhibitor is not PLX7904 or PLX8394.

In some embodiments, the MAPK agonist is vemurafenib. In some embodiments, vemurafenib is administered to the subject at a dose of at least, at most, or about 100, 200, 300, 400, 500, 600, 700, 800, 900, 1000, 1100, 1200, 1300, 1400, 1500, 1600, 1700, 1800, 1900, or 2000 mg, or any range or value derivable therein. In some embodiments, vemurafenib is administered to the subject at a dose of between 200 mg and 300 mg. In some embodiments, vemurafenib is administered to the subject at a dose of between 450 mg and 600 mg. In some embodiments, vemurafenib is administered to the subject at a dose of between 700 mg and 800 mg. In some embodiments, vemurafenib is administered to the subject at a dose of between 900 mg and 1000 mg. In some embodiments, vemurafenib is administered to the subject at a dose of about 960 mg.

B. Epigenetic Modifiers

In some aspects, the ADM inducer is an epigenetic modifier that can alter DNA methylation, histone methylation, acetylation, or interfere with chromatin writers, readers, or erasers able to perturb the transcriptional programs involved in the maintenance of acinar cell identity.

An “epigenetic modifier” refers to an agent that modifies a cell's epigenetic state, e.g., phenotype or gene expression, due to a mechanism other than a change in DNA sequence. The epigenetic state of a cell includes, for example, DNA methylation, histone modifications, and RNA-related silencing.

Non-limiting examples of epigenetic modifiers include: (a) DNA methyltransferases (for example, azacytidine, decitabine or zebularine); (b) histone and protein methyltransferases, including, but not limited to, DOT1L inhibitors such as EPZ004777 (7-[5-Deoxy-5-[[3-[[[[4-(1,1-dimethylethyl)phenyl]amino]carbonyl]amino]propyl](1-methylethyl)amino]-β-D-ribofuranosyl]-7H-pyrrolo[2,3-d]pyrimidin-4-amine), EZH1 inhibitors, EZH2 inhibitors or EPX5687; (c) histone demethylases; (d) histone deacetylase inhibitors (HDAC inhibitors) including, but not limited to, vorinostat, romidepsin, chidamide, panobinostat, belinostat, valproic acid, mocetinostat, abexinostat, entinostat, resminostat, givinostat, or quisinostat; (e) histone acetyltransferase inhibitors (also referred to as HAT inhibitors) including, but not limited to, C-646, (4-[4-[[5-(4,5-Dimethyl-2-nitrophenyl)-2-furanyl]methylene]-4,5-dihydro-3-methyl-5-oxo-1H-pyrazol-1-yl]benzoic acida), CPTH2 (cyclopentylidene-[4-(4′-chlorophenyl)thiazol-2-yl]hydrazine), CTPB (N-(4-chloro-3-trifluoromethyl-phenyl)-2-ethoxy-6-pentadecyl-benzamide), garcinol ((1R,5R,7R)-3-(3,4-Dihydroxybenzyol)-4-hydroxy-8,8-dimethyl-1,7-bis(3-methyl-2-buten-1-yl)-5-[(2S)-5-methyl-2-(1-methylethenyl)-4-hexen-1-yl]bicyclo[3.3.1]non-3-ene-2,9-dione), anacardic acid, EML 425 (5-[(4-hydroxy-2,6-dimethylphenyl)methylene]-1,3-bis(phenylmethyl)-2,4,6(1H,3H,5H)-pyrimidinetrione), ISOX DUAL ([3-[4-[2-[5-(Dimethyl-1,2-oxazol-4-yl)-1-[2-(morpholin-4-yl)ethyl]-1H-1,3-benzodiazol-2-yl]ethyl]phenoxy]propyl]dimethylamine), L002 (4-[O-[(4-methoxyphenyl)sulfonyl]oxime]-2,6-dimethyl-2,5-cyclohexadiene-1,4-dione), NU 9056 (5-(1,2-thiazol-5-yldisulfanyl)-1,2-thiazole), SI-2 hydrochloride (1-(2-pyridinyl)ethanone 2-(1-methyl-1H-benzimidazol-2-yl)hydrazone hydrochloride); or (f) other chromatin remodelers. In some aspects, the epigenetic modifier is vorinostat, romidepsin, belinostat, or panobinostat.

In some aspects, the epigenetic modifier modulates histone modification (e.g., an HDAC modulator). In some aspects, the epigenetic modifier modulates a pathway involving BRD2, BRD4, or EGLN1. In some aspects, the epigenetic modifier is (+)-JQ1; S)-JQ1; belinostat (e.g., PXD101); MS-275 (e.g., entinostat; MS-27-275); vorinostat (e.g., Suberoylanilide hydroxamic acid (SAHA); zolinza); mosetinostat (e.g., MGCD0103); I-BET (e.g., GSK525762A); SB939 (e.g., prinostat; PFI-1); 1215); I-BET151 (e.g., GSK1210151A); IOX2; or derivatives, salts, metabolites, prodrugs, and stereoisomers thereof. In some aspects, the epigenetic modifier is vorinostat. The epigenetic modifier may be a BET inhibitor, such as BRD2, BRD3, BRD4, and/or BRDT inhibitor. In some embodiments, the epigenetic modifier is a BRD4 inhibitor. The BRD4 inhibitor may be, for example, INCB054329, GSK525762A/I-BET762, INCB054329, ABBV-075, OTX015/MK-8628, GSK2820151/I-BET151, PLX51107, ABBV-744, or AZD5153, among others. In certain embodiments, the epigenetic modifier can be a small molecule, peptide, siRNA, sgRNA, PROTAC, or degron. For example, the CRISPR gene-editing system may be used to selectively modify chromatin (e.g., CRISPR dCas9-KRAB).

The compounds described herein may contain one or more asymmetrically-substituted carbon or nitrogen atoms, and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present disclosure can have the (S) or the (R) configuration.

The compounds described herein may also exist in prodrug form. Since prodrugs are known to enhance numerous desirable qualities of pharmaceuticals (e.g., solubility, bioavailability, manufacturing, etc.), the compounds employed in some methods of the disclosure may, if desired, be delivered in prodrug form. Thus, the disclosure contemplates prodrugs of compounds of the present disclosure as well as methods of delivering prodrugs. Prodrugs of the compounds described herein may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a subject, cleaves to form a hydroxy, amino, or carboxylic acid, respectively.

C. Formulations

In some embodiments of the present disclosure, the compounds are included as a pharmaceutical formulation. Materials for use in the preparation of microspheres and/or microcapsules are, e.g., biodegradable/bioerodible polymers such as polygalactin, poly-(isobutyl cyanoacrylate), poly(2-hydroxyethyl-1-glutamine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

Formulations for oral use include tablets containing the active ingredient(s) (e.g., the compounds described herein) in a mixture with non-toxic pharmaceutically acceptable excipients. Such formulations are known to the skilled artisan. Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and anti-adhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the active drug in a predetermined pattern (e.g., in order to achieve a controlled release formulation) or it may be adapted not to release the active drug until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material, such as, e.g., glyceryl monostearate or glyceryl distearate may be employed.

D. Cell Targeting Moieties

In some aspects, the present disclosure provides compounds conjugated directly or through linkers to a cell targeting moiety, such as PROTAC and degrons, and/or agents delivered through vesicles such as exosomes and liposomes. In some embodiments, the conjugation/inclusion of the compound to a cell targeting moiety/vesicle increases the efficacy of the compound in treating a disease or disorder. Cell targeting moieties/vesicles according to the embodiments may be, for example, an antibody, a growth factor, a hormone, a peptide, an aptamer, a drug, a small molecule, a hormone, an imaging agent, cofactor, cytokine, or vesicles (e.g., exosomes and/or liposomes. In some embodiments, the compounds of the present disclosure may be used in conjugates with an antibody for a specific antigen that is expressed by a cancer cell but not in normal tissues. In some embodiments, compounds of the present disclosure may be used in conjugates with an antibody for a specific antigen that is expressed by pancreatic cells but not by other cell types.

Since a large number of cell surface receptors have been identified in hematopoietic cells of various lineages, ligands or antibodies specific for these receptors may be used as cell-specific targeting moieties. IL-2 may also be used as a cell-specific targeting moiety in a chimeric protein to target IL-2R+ cells. Alternatively, other molecules such as B7-1, B7-2 and CD40 may be used to specifically target activated T cells. Furthermore, B cells express CD19, CD40 and IL-4 receptor and may be targeted by moieties that bind these receptors, such as CD40 ligand, IL-4, IL-5, IL-6 and CD28. The elimination of immune cells such as T cells and B cells is particularly useful in the treatment of lymphoid tumors.

Other cytokines that may be used to target specific cell subsets include the interleukins (IL-1 through IL-15), granulocyte-colony stimulating factor, macrophage-colony stimulating factor, granulocyte-macrophage colony stimulating factor, leukemia inhibitory factor, tumor necrosis factor, transforming growth factor, epidermal growth factor, insulin-like growth factors, and/or fibroblast growth factor (Thompson (ed.), 1994, The Cytokine Handbook, Academic Press, San Diego). In some aspects, the targeting polypeptide is a cytokine that binds to the Fn14 receptor, such as TWEAK.

A skilled artisan recognizes that there are a variety of known cytokines, including hematopoietins (four-helix bundles) [such as EPO (erythropoietin), IL-2 (T-cell growth factor), IL-3 (multicolony CSF), IL-4 (BCGF-1, BSF-1), IL-5 (BCGF-2), IL-6 IL-4 (IFN-β2, BSF-2, BCDF), IL-7, IL-8, IL-9, IL-11, IL-13 (P600), G-CSF, IL-15 (T-cell growth factor), GM-CSF (granulocyte macrophage colony stimulating factor), OSM (OM, oncostatin M), and LIF (leukemia inhibitory factor)]; interferons [such as IFN-7, IFN-α, and IFN-0); immunoglobin superfamily (such as B7.1 (CD80), and B7.2 (B70, CD86)]; TNF family [such as TNF-α (cachectin), TNF-β (lymphotoxin, LT, LT-α), LT-β, CD40 ligand (CD40L), Fas ligand (FasL), CD27 ligand (CD27L), CD30 ligand (CD30L), and 4-1BBL)]; and those unassigned to a particular family [such as TGF-β, IL 1α, IL-1, IL-1 RA, IL-10 (cytokine synthesis inhibitor F), IL-12 (NK cell stimulatory factor), MIF, IL-16, IL-17 (mCTLA-8), and/or IL-18 (IGIF, interferon-7 inducing factor)]. Furthermore, the Fc portion of the heavy chain of an antibody may be used to target Fc receptor-expressing cells such as the use of the Fc portion of an IgE antibody to target mast cells and basophils.

Furthermore, in some aspects, the cell-targeting moiety may be a peptide sequence or a cyclic peptide. Examples, cell- and tissue-targeting peptides that may be used according to the embodiments are provided, for instance, in U.S. Pat. Nos. 6,232,287; 6,528,481; 7,452,964; 7,671,010; 7,781,565; 8,507,445; and 8,450,278, each of which is incorporated herein by reference.

Thus, in some embodiments, cell targeting moieties are antibodies or avimers. Antibodies and avimers can be generated against virtually any cell surface marker thus, providing a method for targeted to delivery of GrB to virtually any cell population of interest. Methods for generating antibodies that may be used as cell targeting moieties are detailed below. Methods for generating avimers that bind to a given cell surface marker are detailed in U.S. Patent Publications Nos. 2006/0234299 and 2006/0223114, each incorporated herein by reference.

Additionally, it is contemplated that the compounds described herein may be conjugated to a nanoparticle or other nanomaterial. Some non-limiting examples of nanoparticles include metal nanoparticles such as gold or silver nanoparticles or polymeric nanoparticles such as poly-1-lactic acid or poly(ethylene) glycol polymers. Nanoparticles and nanomaterials which may be conjugated to the instant compounds include those described in U.S. Patent Publications Nos. 2006/0034925, 2006/0115537, 2007/0148095, 2012/0141550, 2013/0138032, and 2014/0024610 and PCT Publication No. 2008/121949, 2011/053435, and 2014/087413, each incorporated herein by reference.

### II. METHODS OF USE

Embodiments of the present disclosure concern methods for the use of one or more ADM inducers for treating or preventing pancreatitis or pancreatic cancer. The disclosed methods may include administering to the subject a therapeutically effective amount of the one or more ADM inducers, thereby treating or preventing pancreatitis or pancreatic cancer in the subject. In some embodiments, disclosed is a method for treatment of pancreatitis comprising administering an effective amount of an ADM inducer to a subject. In some embodiments, disclosed is a method for preventing pancreatic cancer comprising administering an effective amount of an ADM inducer to a subject.

“Treating” or treatment of a disease or condition refers to executing a protocol, which may include administering one or more drugs to a patient, in an effort to alleviate signs or symptoms of the disease. Desirable effects of treatment include decreasing the rate of disease progression, ameliorating or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

The term “effective,” as that term is used in the specification and/or claims, means adequate to accomplish a desired, expected, or intended result. “Effective amount,” “therapeutically effective amount” or “pharmaceutically effective amount” when used in the context of treating a patient or subject with a compound means that amount of the compound which, when administered to a subject or patient for treating or preventing a disease, is an amount sufficient to affect such treatment or prevention of the disease.

A. Treatment of Pancreatitis

Aspects of the present disclosure are directed to compositions and methods for treatment of pancreatitis. In some embodiments, disclosed is a method for treating a subject for pancreatitis comprising administering one or more ADM inducers to the subject. In some embodiments, the pancreatitis is acute pancreatitis. In some embodiments, the pancreatitis is chronic pancreatitis. In some embodiments, a subject of the disclosure is suspected of having pancreatitis. In some embodiments, a subject of the disclosure has been diagnosed with pancreatitis. A subject may be diagnosed with pancreatitis using tests and diagnostic methods known in the art. For example, a subject may be determined to have pancreatitis by testing the subject for one or more symptoms of pancreatitis. In another example, a subject is determined to have pancreatitis by detecting an increased level of one or more pancreatic enzymes (e.g., amylase, lipase) in the subject relative to a control or healthy subject.

B. Treatment and Prevention of Cancer

Aspects of the present disclosure are directed to compositions and methods for treatment and prevention of cancer. The term “cancer,” as used herein, may be used to describe a solid tumor, metastatic cancer, or non-metastatic cancer. In certain embodiments, the cancer may originate in the blood, bladder, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus.

In some embodiments, disclosed are methods for treating or preventing pancreatic cancer. In some embodiments, disclosed is a method for preventing pancreatic cancer. In some embodiments, the pancreatic cancer is pancreatic ductal adenocarcinoma (PDAC). Methods for preventing pancreatic cancer may comprise administration of one or more ADM inducers to a subject at risk of developing pancreatic cancer. In some embodiments, the subject has not been diagnosed with pancreatic cancer.

C. Pharmaceutical Formulations and Routes of Administration

Where clinical applications are contemplated, it will be necessary to prepare pharmaceutical compositions in a form appropriate for the intended application. In some embodiments, such formulation with the compounds of the present disclosure is contemplated. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present disclosure comprise an effective amount of the vector to cells, dissolved or dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present disclosure, its use in therapeutic compositions is contemplated. Supplementary active ingredients also can be incorporated into the compositions.

The active compositions of the present disclosure may include classic pharmaceutical preparations. Administration of these compositions according to the present disclosure will be via any common route so long as the target tissue is available via that route. Such routes include oral, nasal, buccal, rectal, vaginal or topical route. Alternatively, administration may be by orthotopic, intradermal, subcutaneous, intramuscular, intraperitoneal, or intravenous injection. Such compositions would normally be administered as pharmaceutically acceptable compositions, described supra.

The active compounds may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For oral administration the compounds described herein may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present disclosure may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 mL of hypodermoclysis fluid or injected at the proposed site of infusion, (see for example, “Remington's Pharmaceutical Sciences,” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by the appropriate regulatory agencies for the safety of pharmaceutical agents.

In particular, the compositions that may be used are disclosed herein. The compositions described above are preferably administered to a mammal (e.g., rodent, human, non-human primates, canine, bovine, ovine, equine, feline, etc.) in an effective amount, that is, an amount capable of producing a desirable result in a treated subject (e.g., inducing ADM). Toxicity and therapeutic efficacy of the compositions utilized in methods of the disclosure can be determined by standard pharmaceutical procedures. As is well known in the medical and veterinary arts, dosage for any one animal depends on many factors, including the subject's size, body surface area, body weight, age, the particular composition to be administered, time and route of administration, general health, the clinical symptoms of the infection or cancer and other drugs being administered concurrently. A composition as described herein is typically administered at a dosage that induces pharmacological effects (e.g., ADM), as assayed by identifying a reduction in hematological parameters (complete blood count—CBC, enzymes and inflammatory indexes), amelioration in clinical (pain) or imaging parameters (edema, vascularization, size). In some embodiments, amounts of the compounds used to induce the desired effects is calculated to be from about 0.01 mg to about 10,000 mg/day. In some embodiments, the amount is from about 1 mg to about 1,000 mg/day. In some embodiments, these dosings may be reduced or increased based upon the biological factors of a particular patient such as increased or decreased metabolic breakdown of the drug or decreased uptake by the digestive tract if administered orally. Additionally, the compounds may be more efficacious and thus a smaller dose is required to achieve a similar effect. Such a dose is typically administered once a day for a few weeks or until sufficient clinical improvement has been achieved.

The therapeutic methods of the disclosure (which include prophylactic treatment) in general include administration of a therapeutically effective amount of the compositions described herein to a subject in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, marker (as defined herein), family history, and the like).

D. Combination Therapies

Certain embodiments of the present disclosure provide for the administration or application of one or more secondary forms of therapies for the treatment or prevention of a disease. For example, the disease may be a hyperproliferative disease, such as cancer. In another example, the disease is pancreatitis.

The secondary form of therapy may be administration of one or more secondary pharmacological agents that can be applied in the treatment or prevention of cancer. If the secondary therapy is a pharmacological agent, it may be administered prior to, concurrently, or following administration of the present compounds.

The interval between the administration of the present compounds and the secondary therapy may be any interval as determined by those of ordinary skill in the art. For example, the interval may be minutes to weeks. In embodiments where the agents are separately administered, one would generally ensure that a long period of time did not expire between the time of each delivery, such that each therapeutic agent would still be able to exert an advantageously combined effect on the subject. For example, the interval between therapeutic agents may be about 12 h to about 24 h of each other and, more preferably, within about 6 hours to about 12 h of each other. In some situations the time period for treatment may be extended, however, where several days (2, 3, 4, 5, 6 or 7) to several weeks (1, 2, 3, 4, 5, 6, 7 or 8) lapse between the respective administrations. In some embodiments, the timing of administration of a secondary therapeutic agent is determined based on the response of the subject to the nanoparticles.

Various combinations may be employed. For the example below a MAPK agonists is “A” and an anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present disclosure to a patient will follow general protocols for the administration of such compounds, taking into account the toxicity, if any, of the agents. Therefore, in some embodiments there is a step of monitoring toxicity that is attributable to combination therapy. It is expected that the treatment cycles may be repeated. It also is contemplated that various standard therapies, as well as surgical intervention, may be applied in combination with the described therapy.

In specific aspects, it is contemplated that a standard therapy will include anti-inflammatory and/or analgesic agents for pancreatitis and may be employed in combination with the inducers of ADM as described herein.

The skilled artisan will understand that immunotherapies may be used in combination or in conjunction with methods of the embodiments (e.g., ADM inducers), such as to eradicate the clonal expansion of KRAS mutated cells. In the context of cancer prevention, immunotherapeutics may rely on the use of immune effector cells and molecules to target, destroy and/or limit the expansion and counteract the positive selection of KRAS mutated cells. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells.

In one aspect of immunotherapy, the tumor cell may bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies that may be used are immune adjuvants, e.g., Mycobacterium bovis, Plasmodium falciparum, dinitrochlorobenzene, and aromatic compounds; cytokine therapy, e.g., interferons α, β and γ, IL-1, GM-CSF, and TNF; gene therapy, e.g., TNF, IL-1, IL-2, and p53; and monoclonal antibodies, e.g., anti-CD20, anti-ganglioside GM2, and anti-p185. It is contemplated that one or more anti-cancer therapies may be employed with the antibody therapies described herein.

In some embodiments, the immunotherapy may be an immune checkpoint inhibitor. Immune checkpoints are molecules in the immune system that either turn up a signal (e.g., co-stimulatory molecules) or turn down a signal. Inhibitory checkpoint molecules that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies. Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example, it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156, can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof. In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesions such as described in U.S. Pat. No. 8,329,867, incorporated herein by reference.

It is contemplated that other agents may be used in combination with certain aspects of the present embodiments to improve the therapeutic efficacy of treatment. Further examples can therefore be contemplated. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### III. EXAMPLES

The following examples are included to demonstrate certain embodiments of the invention. It should be appreciated by those of skill in the art that the techniques disclosed in the examples which follow represent techniques discovered by the inventor to function well in the practice of the invention, and thus can be considered to constitute certain modes for its practice. However, those of skill in the art should, in light of the present disclosure, appreciate that many changes can be made in the specific embodiments which are disclosed and still obtain a like or similar result without departing from the spirit and scope of the invention.

### Example 1—Epithelial Memory of Resolved Inflammation Limits Tissue Damage while Promoting Pancreatic Tumorigenesis

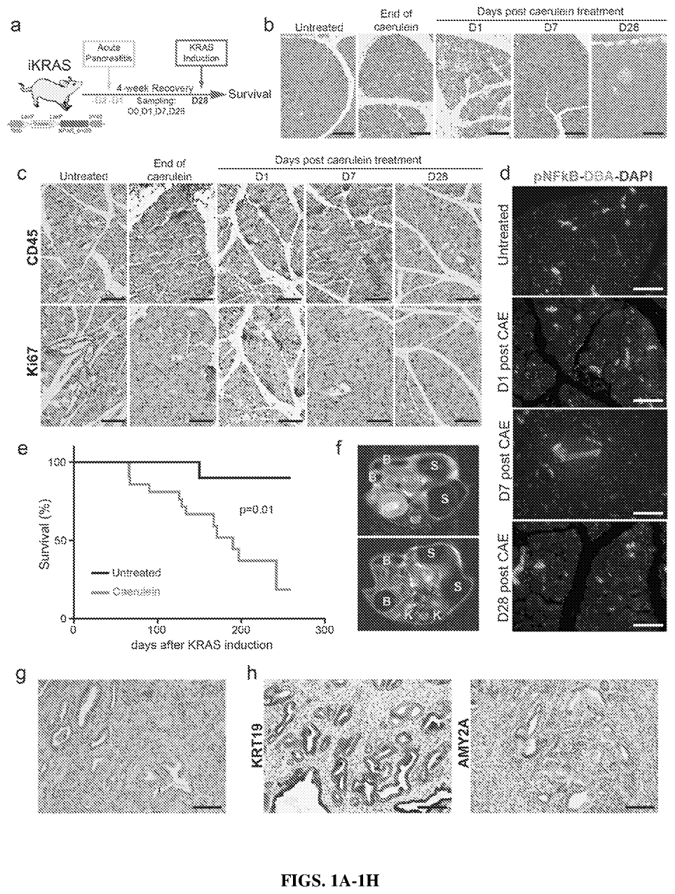

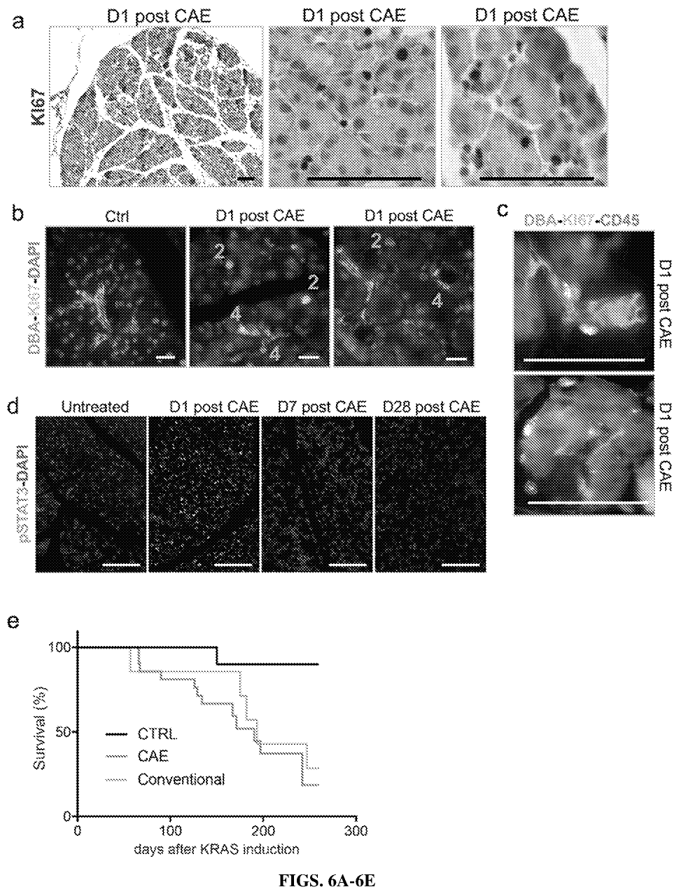

Transient inflammation promotes tumorigenesis long after its resolution: To investigate the long-term effect of inflammation on the transformation of normal pancreatic epithelial cells, caerulein (hereafter CAE), a decapeptide analog of cholecystokinin (Bowie, 2013), was used to trigger damage and subsequent inflammation in a well-characterized PDAC mouse model in which oncogenic KRAS^{G12D} expression is induced in the pancreas via doxycycline administration (iKRAS model) (Ying et al., 2012; Viale et al., 2014; Kapoor et al., 2014). To avoid major confounding effects linked to chronic CAE administration, such as stromal and microenvironment remodeling, a protocol of acute inflammation was used consisting of a 2-day CAE administration (Mayerle, 2013) (FIG. 1A). Immediately after CAE administration a transient pancreatic inflammation was observed, with edema and inter/intra-lobular infiltration of inflammatory cells, followed by a rapid restoration of tissue integrity by day 7 (FIG. 1B). Immunostaining was consistent with the histological analysis, revealing that the inflammatory infiltration (CD45+ cells) and proliferation (Ki67 staining) present at day 1 (D1) post-CAE treatment, returned to pre-CAE levels after 7 days (FIG. 1C and FIG. 6A-C), indicating a complete reestablishment of histological resolution. In addition, strong nuclear staining of the major transcriptional activators of inflammation NF-kB (Hayden et al., 2012) and STAT3 (Pardoll and Jove, 2009) was observed in epithelial and stromal cells immediately following the induction of pancreatitis. The normalization of these two transcription factors, together with the recovery of the normal pancreatic histology suggested that the inflammatory response was extinguished one week post-CAE treatment (FIG. 1D and FIG. 6D). Therefore, the effects of oncogenic KRAS was explored after resolution of this single inflammatory event by inducing its expression at 28 days after pancreatitis and monitoring tumor development (FIG. 1A). CAE-treated mice developed tumors with high penetrance and succumbed to disease earlier than untreated animals (median survival of 190 days in CAE-treated versus undefined in untreated animals; p=0.01) (FIG. 1E). This observation was also confirmed by nuclear magnetic resonance (FIG. 1F) and histological analysis (FIG. 1G-H). Importantly, the survival of the animals recovered from inflammation overlapped the survival of mice in which KRAS was activated prior to the induction of inflammation (FIG. 6E). Overall, these data show that transient inflammatory events have persistent effects on normal epithelial cells and can cooperate with oncogene activation long after their resolution.

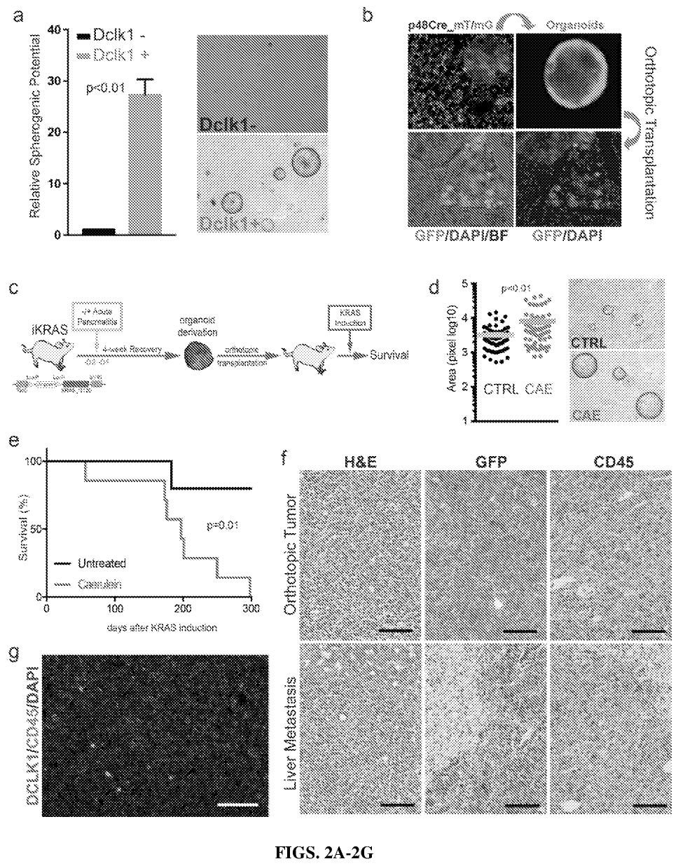

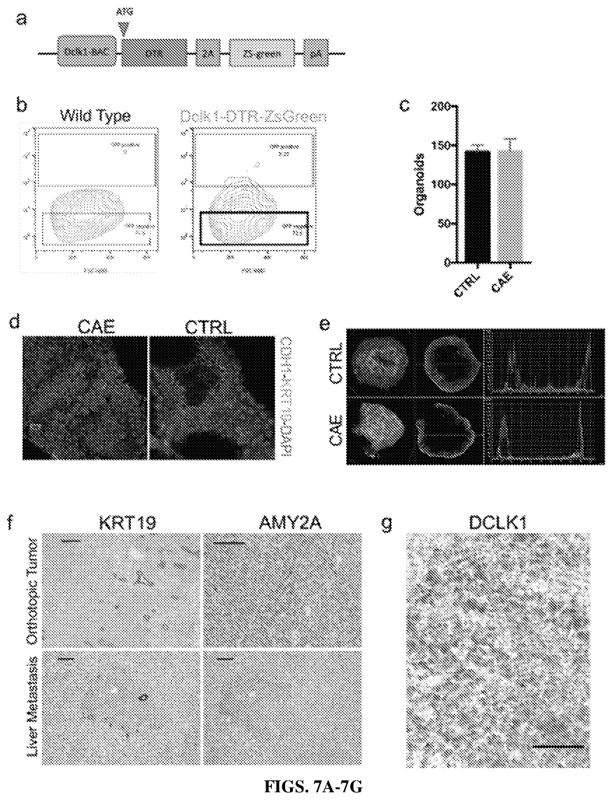

Long-term effects of resolved inflammation are cell autonomous: To determine whether differences in outcome between CAE-treated and untreated animals resulted from epithelial cell-autonomous effects or from the influence of enduringly activated stroma, epithelial cultures were established from pancreata of mice 4 weeks after acute CAE treatment, as well as from untreated control animals. Because two-dimensional (2D) cultures derived from wild-type pancreas undergo a limited expansion in vitro, epithelial cells were cultured as 3D organoids, a well-established culturing system maintained by a population of progenitor cells able to sustain pancreas regeneration in vivo (Westphalen et al., 2016). It was first confirmed that under these experimental conditions, epithelial organoids derive from progenitor cells. Pancreatic progenitors have been described to be positive for Doublecortin-Like Kinase 1 (DCLK1). Therefore, using a mouse model in which the green fluorescent protein is expressed under the control of the Dclk1 promoter (Dclk1-DTR-ZsGreen) (FIG. 7A), it was found that the only pancreatic cells able to generate organoids were in the ZsGreen positive fraction (FIGS. 2A and 7B), as previously reported (Westphalen et al., 2016).

To further corroborate that organoids represent a source of functional pancreatic progenitors, their ability to regenerate normal pancreatic tissue upon transplantation was assessed. For this purpose, in order to trace their epithelial origin, GFP positive organoids derived from P48-Cre;R26-mT/mG animals were transplanted orthotopically in syngeneic recipients in which pancreata were damaged by CAE treatment. Four weeks after implantation, GFP-positive lobules were clearly detected in transplanted animals (FIG. 2B). Next, organoids were derived from animals that recovered from inflammation and matched controls (FIG. 2C). Although numerically and morphologically similar (FIG. 7C-D), organoids derived from mice pre-exposed to inflammation showed an increased size with respect to controls (FIGS. 2D and 7E), suggesting that epithelial cells previously exposed to inflammation can more efficiently expand in vitro. After 5 weeks in culture, iKRAS organoids were orthotopically transplanted into inflammation-naïve recipients, and KRAS was induced (FIG. 2C). Mice that received organoids derived from CAE-treated pancreata developed tumors with higher penetrance compared to controls (FIG. 2E). These tumors were highly aggressive, as shown by both liver secondary localizations and poorly differentiated histology (FIG. 2F, left panels). The focal positivity for markers of pancreatic exocrine differentiation, such as CK19 and amylase (FIG. 7F), the positivity for GFP and the exclusion of CD45 immunoreactivity cumulatively confirmed the pancreatic origin of these tumors (FIG. 2F, central-right panels). Notably, the extensive positivity for Dclk1 (FIG. 2G and FIG. 7G), accounting for lack of differentiation, suggests tumors in this experimental setting are derived from the transformation of the progenitor cells that maintain the pancreatic organoids.

These data indicate that the long-lasting epithelial modifications that cooperate with oncogenic signaling are cell-autonomous and maintained over time by a pool of progenitor cells able to sustain pancreatic regeneration and tumorigenesis.

Transient inflammatory events induce sustained transcriptomic deregulation in epithelial cells: Next, a transcriptomic analysis of post-inflammation and control wild-type organoids was performed 9 weeks after CAE treatment, which included 4 weeks of recovery in vivo prior to 5 weekly passages ex vivo. 441 upregulated and 416 downregulated genes (FDR≤0.05, Log 2 FC≥0.8) were identified (FIG. 3A). Gene Set Enrichment (GSEA) and Ingenuity Pathway (IPA) analyses showed the activation of gene expression programs involved in development, cell migration, wound healing and cancer specifically in organoids derived from CAE-treated animals (FIG. 3B,C). Key signaling pathways involved in PDAC, such as RAS and p53, were deregulated as well (FIG. 3B). Notably, a set of genes (Development/Progression), coregulated during pancreatic embryonic development and tumorigenesis (Reichert et al., 2013), was significantly enriched in organoids derived from inflamed pancreata (FIG. 3B). These findings support the notion that epithelial cells maintain an adaptive response to tissue damage which includes the sustained activation of multiple gene expression programs, including embryonic programs reactivated during cancer progression.

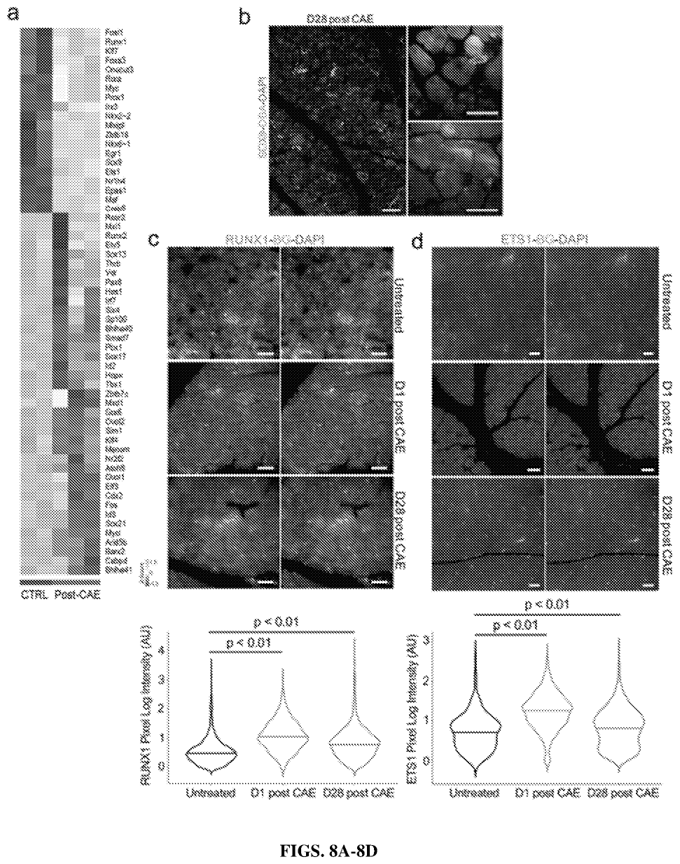

To identify the regulatory networks responsible for this persistent transcriptional signature, organoid expression data was first interrogated and 59 transcription factors (20 upregulated and 39 downregulated) were identified whose expression was persistently altered after the acute inflammatory event (FIG. 8A). In addition to transcription factors involved in proliferation, such as E2f family members, transcription factors, such as Sox9, Runx1, Ets1 and Myc, were found that are important players in tumor progression and are known to be specifically relevant in pancreatic cancer (Scheitz et al., 2012; Dittmer, 2015; Mazur et al., 2015; Genovese et al., 2017).

To obtain a more comprehensive description of the sustained regulatory changes induced by a previous inflammation, ChIP-sequencing experiments were carried out on paired organoids from inflamed and non-inflamed pancreata using antibodies for H3K27Ac, which detects active enhancer and promoter regions. Analogously to gene expression changes, a large number of persistent differences were found in histone acetylation (3,520 hyperacetylated and 2,913 hypoacetylated regions, 90% of which were located distally from gene promoters) between organoids from untreated and CAE-treated mice (FIG. 3D).

Next, DNA sequence motifs statistically over-represented in the promoters of deregulated genes relative to all other RefSeq genes, as well as in the distal differentially acetylated regions were identified (FIG. 3E). In the promoter regions, an overrepresentation of motifs recognized by EGR1 was found, a transcriptional regulator of the early growth response gene family (Thiel and Cibelli, 2002), whose expression was also persistently upregulated in organoids from CAE-treated mice. Moreover, an over-representation of motifs recognized by SOX and ONECUT family members was found in the hypoacetylated regions, while motifs for FOXA3 and NK-related factors, like NKX2-2, NKX6-1 and BARX2, were over-represented in the hyperacetylated set (FIG. 3E).

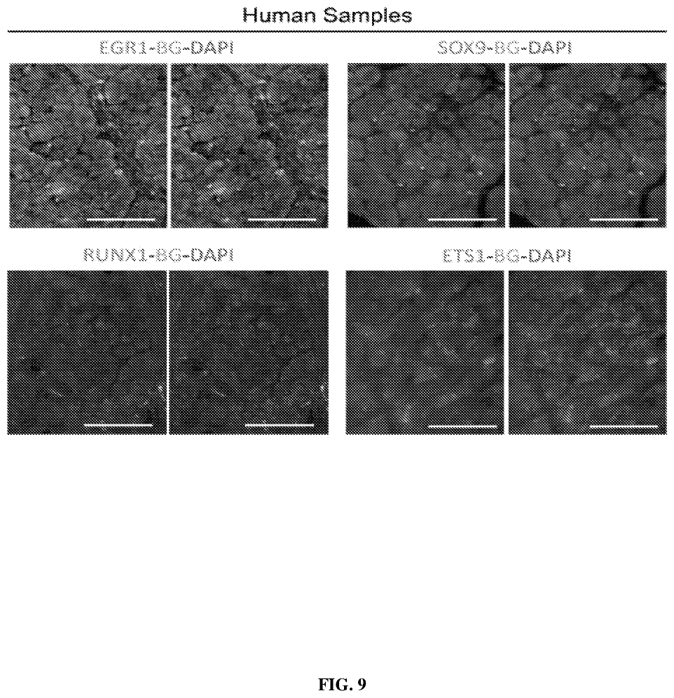

To validate the possible role of the transcription factors identified from the previous analyses, immunostaining experiments were carried out that showed, that while EGR1, ETS1, RUNX1 and SOX9 were not expressed in normal pancreas, or expressed exclusively in ductal cells like SOX9, they were instead highly expressed in the nucleus of the vast majority of the epithelial cells after CAE administration (D1). Albeit less intense, their ectopic expression persisted in acinar cells at late time points (D28) (FIGS. 3F and 8B-D). Notably, the same transcription factors were upregulated in samples of human chronic pancreatitis analyzed by immunostaining (FIG. 9).

Taken together, these data demonstrate that after normal pancreatic epithelial cells histologically recover from a transient episode of inflammation, they acquire a long-lasting adaptive response maintained by a persistent transcriptional reprogramming.

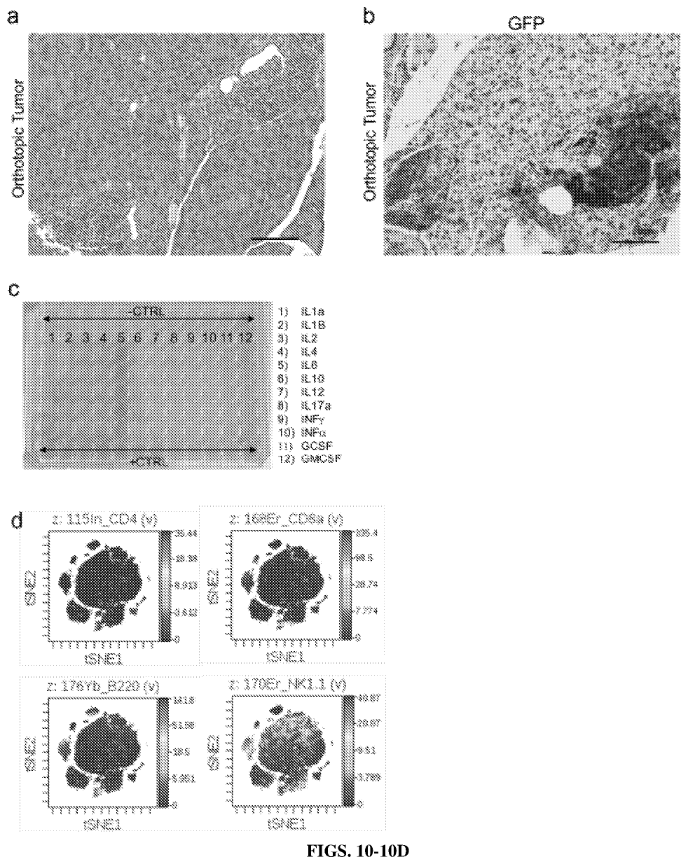

IL-6 mediates epithelial reprogramming during inflammatory events: To test whether epithelial reprogramming is dependent on the activity of inflammatory cells, epithelial organoids derived were cultured from iKRAS pancreas with medium conditioned by CD45-positive cells isolated from acute pancreatitis. After one week, organoids were transferred to conventional medium and maintained in culture for additional 4 weeks to minimize acute effects of cytokine exposure (FIG. 4A). Organoids exposed to CD45-conditioned medium or control organoids were then orthotopically transplanted into recipient mice, and KRAS expression was induced. Only mice injected with CD45-conditioned cells developed tumors that histologically resembled those obtained from transplantation of organoids derived from CAE-treated pancreas (FIGS. 4B and 10A). The epithelial origin of these tumors was confirmed by positivity for the GFP marker (FIG. 10B).

This experiment confirms that epithelial cells undergo reprogramming ex vivo through soluble molecules released by inflammatory cells that mediate inflammation-induced changes in the pancreatic epithelium. ELISA analysis of CD45-conditioned medium revealed the presence of high levels of IL-6 and G-CSF (FIGS. 4C and 10C). Since the G-CSF receptor is not expressed in pancreatic cells according to the data set, IL-6 was considered, whose role in PDAC progression is supported by a large body of evidences (Grivennikov et al., 2009; Karin and Clevers, 2016; Fukuda et al., 2011; Lesina et al., 2011), as the most likely player. Exposure of organoids to CD45-conditioned medium, as well as to recombinant Hyper-IL6, a potent chimeric molecule able to engage gp130 trans-signaling, confirmed a strong induction of Stat3 phosphorylation at Tyr705 (FIG. 4D). Consistently, in vivo immunostaining analysis revealed the co-detection of IL-6-positive infiltrating cells and nuclear phospho-STAT3 signal in virtually all acinar cells in pancreatic samples immediately after CAE-treatment (D1) (FIG. 4E).

Mass cytometry immunophenotyping of CD45 cells recruited to the pancreas upon acute CAE exposure (D1) revealed a massive infiltration of macrophages (CD68+, F4/80+, CD11+) (FIG. 4F) with only a marginal contribution of lymphoid cells (CD4, CD8, B220, NK1.1) (FIG. 10D). Furthermore, tSNE representation of immunoreactivity for IL-6 completely overlapped with CD68, F4/80 and CD11 markers identifying macrophages as the major source of IL-6 production in vivo (FIG. 4F). To definitively demonstrate that IL-6 is a mediator of the epithelial reprogramming, IL-6-treated organoids were measured for the expression of key transcription factors found deregulated in vivo upon pancreatitis. Immunoblotting for EGR1, RUNX1, ETS1 and SOX9 revealed their strong upregulation after exposure of organoids to Hyper-IL6 for 24 hours (FIG. 4G).

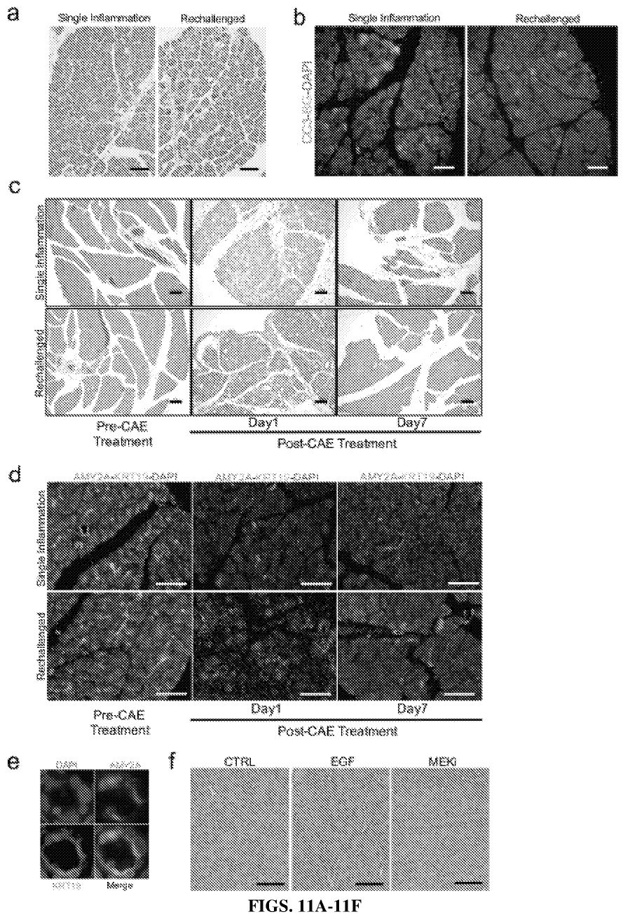

Acinar to ductal metaplasia is facilitated by epithelial memory to limit tissue damage: As any adaptive process, epithelial memory of previous inflammation should confer an evolutionary advantage. Because of the deregulation of ectopic transcription factors mainly in the acinar compartment in vivo, one possibility is that such memory provides a defense mechanism in case of recurring inflammatory events that would otherwise result in the repeated release of pancreatic enzymes and cumulative tissue damage. To understand how a discrete inflammatory episode can influence subsequent inflammatory events, animals who had recovered from CAE-induced acute pancreatitis were rechallenged with a second inflammation (FIG. 5A). Early evaluation of pancreatic enzymes (FIG. 5B) as well as lactate dehydrogenase (LDH, a marker of cell lysis) (FIG. 5C) in the blood of wild-type mice at 24 h after pancreatitis induction, revealed that both enzymes in the rechallenged group were almost comparable to control animals. This result suggests that a sustained adaptive response triggered by the first inflammatory event attenuated pancreatic damage induced by a second acute inflammation. Indeed, at the histological level only the pancreata of animals receiving the first inflammatory trigger showed the presence of extensive acinar damage (FIG. 5D, left panels), as further confirmed by immunostaining for cleaved caspase 3 (CC3) (FIG. 5D, right panels). Unexpectedly, the pancreata of rechallenged animals responded to the second inflammatory event by undergoing an extensive acinar-to-ductal metaplasia (ADM) that was completely manifested within 48 hours post-CAE administration (FIG. 5E and FIG. 11C-E). Moreover, the ADM event was completely resolved by day 7 post-CAE administration, as demonstrated by the full recovery of functional pancreatic tissue (FIG. 11C, D).

Thus, the sustained adaptive response triggered in the pancreatic epithelium by an acute inflammatory event resulted in a markedly attenuated response to subsequent inflammatory episodes. Such decreased tissue damage was accompanied by the rapid dedifferentiation of acinar cells that lasted for the length of the stimulus and from which the tissue promptly and apparently completely recovered. To explore the hypothesis that ADM is a physiologic, fast and reversible adaptation mediated by epithelial memory that limits the detrimental effects of repeated pancreatitis, the effects of pharmacological modulation of ADM was evaluated in iKRAS animals subjected to repeated inflammation. Because ADM is mediated by the activation of MAPK signaling (Halbrook et al., 2017; Shi et al., 2013), ADM formation was counteracted or promoted with a clinical MEK1-2 inhibitor (Trametinib) or EGF (a MAPK activator), respectively (FIG. 5A). Mice that were pretreated with EGF before and during CAE rechallenge had a further increase of ADM formation with respect to control mice rechallenged with CAE alone (˜3-fold relative area increase, p<0.01) (FIG. 5F, 5G and FIG. 11F) with decreased tissue damage as indicated by CC3 immunostaining (˜8-fold, p<0.01) (FIG. 5F, 5H). Conversely, animals pre-treated with Trametinib developed minimal ADM (FIG. 5F, 5G and FIG. 11F) accompanied by a very severe pancreatitis with massive apoptosis and extensive acinar loss (FIG. 5F-H). Taken together, these data support a model in which sustained epithelial adaptation in response to inflammation involves the facilitation of ADM. By blocking the production of acinar zymogens during sequential inflammatory events, facilitated ADM provides strong protection from tissue damage.

Because ADM has protective effects against pancreatic damage, it was posited that selection of mutations that confer constitutive activation of MAPK signaling, such as mutations of KRAS, may be beneficial and under strong evolutionary pressure. Toward an initial evaluation of this possibility, the impact of inducing mutant KRAS prior to a second inflammatory event was studied. Indeed, in animals with epithelial memory, constitutive activation of KRAS signaling prior to the second CAE exposure resulted in massive ADM (FIG. 5F, 5G) and virtually no tissue damage (FIG. 5F, 5G).

In light of these strong evidences, the use of pharmacological modulation of ADM through the activation of MAPKs is proposed herein as a means to suppress tissue damage, resolve inflammation and prevent the acquisition of mutations of KRAS that would lead to development of pancreatic cancer during repeated pancreatitis.

To demonstrate that the approach based on the pharmacological positive modulation of MAPKs signaling pathways, a mechanism evolved in animals to minimize damage induced by pancreatitis, is superior to current approaches to limit pancreatic inflammation, mice were pretreated with sulindac, a potent anti-inflammatory drug (60 mg/kg, i.p., one injection a day starting 24 hrs before caerulein treatment for a total of four days) or MAPKs agonist EGF (1.2 mg/kg, i.p., two injections a day for a total of four days) before induction of pancreatitis through caerulein administration. As shown in FIG. 12, infiltration of CD45 cells, a well-established marker of active inflammation, was almost completely suppressed by EGF pretreatment demonstrating the validity of the hypothesis and the superiority of the present approach with respect to conventional agents, such as sulindac.

Finally, to definitely demonstrate the translational applicability of the findings the impact of ADM inducers, such as small molecule MAPK activators and epigenetic modifiers, on pancreatitis was evaluated.

MAPK activators: No small-molecule drugs designed to be selective and potent activators of MAPK signaling are currently commercially available. The only ones reported to have paradoxal activity as MAPK activators are the RAF inhibitors when specifically applied to RAF wild-type genetic contexts (Joseph et al., 2010; Carnahan et al., 2010). Indeed, when wild-type mice were pre-treated with Vemurafenib (PLX4032, a selective inhibitor of V600E mutant BRAF) before and during the induction of pancreatitis (75 mg/kg, oral gavage, two injections a day starting 48 hrs before caerulein treatment for a total of eight injections), a prominent activation of the MAPK signaling was observed in the pancreatic tissue, as validated by increase in ERK phosphorylation. Consistent with the observation that ADM might ameliorate the detrimental effects of pancreatitis, a massive reduction in immune cell infiltrates was also detected as demonstrated by CD45 staining and quantification (FIG. 13A-13B). Once again, the pharmacological modulation of ADM through MAPK activation proved to be a valuable therapeutic option to minimize tissue damage and resolve pancreatic inflammation, potentially abrogating selective pressure to acquire tumor-initiating KRAS mutations. Specifically, Vemurafenib and other RAF inhibitors constitute first-generation small-molecule MAPK activators with clinical-grade potential to resolve pancreatitis with already acceptable safety profiles.

Epigenetic modifiers: Recently, the emerging role of epigenetic regulation has led to the development of a wide spectrum of small molecules targeting selectively bromodomain and extra-terminal (BET) protein family which are currently under evaluation in preclinical model of hematological malignancies and solid tumors (Asangani et al., 2014; Delmore et al., 2011; Filippakopoulos et al., 2010). Leveraging the persistent chromatin modifications induced by an acute inflammatory event, the possibility of limiting the detrimental effects of repeated pancreatitis was tested through the administration of INCB054329, a BRD4 inhibitor currently in phase I-II clinical studies in patients with advanced malignancies (Falchook et al., 2019). To assess the ability of the compound to induce ADM in a context of repeated pancreatitis 40 mg/kg b.i.d of INCB054329 was administered by oral gavage before and during Caerulein treatment. The histological evaluation of pancreatic tissues collected at 24 hours after the end of Caerulein injections revealed a massive induction of ADM in BRD4i (INCB054329) treated mice. Consistent with the previous data, the extended ADM was accompanied by a dramatic reduction of inflammatory infiltration as demonstrated by CD45 immunostaining, emphasizing the protecting role of ADM in preserving the tissue integrity upon inflammatory events.

### Example 2—Materials and Methods

Mice: iKRAS mouse model (TetO-LSL-KrasG^{12D}; ROSA26-LSLrtTa-IRES-GFP; p48_Cre) was generated as previously described (Ying et al., 2012). DCLK1-DTR-zsGreen mouse model was generated in Dr. Timothy Craig Wang's lab as described here. The DTR-2A-Zsgreen-pA-FrtNeoFrt cassette was ligated into a pL451 plasmid. A BAC clone RP23-283D6 containing an approximately 50-kb 5′ sequence of the Dclk1 gene-coding region (CHORI) was isolated and transferred into SW105-competent cells. The correct sequence was confirmed by using restriction enzyme digestion and PCR in the region of interest. The purified DTR-2A-Zsgreen-pA-FrtNeoFrt with a probe containing a 75-bp sequence homologous to the BAC sequence directly upstream and downstream of the ATG in exon 2 of mouse Dclk1 gene was electroporated into SW105 Dclk1-BAC-containing cells. BAC DNA was isolated, linearized, and then microinjected into the pronucleus of fertilized CBA×C57BL/6J oocytes at the Columbia University Transgenic Animal Core facility. One positive founder was identified and backcrossed to C57BL/6J mice.

B6.129(Cg)-Gt(ROSA)26Sor^{tm4(ACTB-tdTomato,-EGFP)Luo}/J (referred to as R26_mT/mG) mice were generated in Dr. Liqun Luo's lab and purchased from The Jackson Laboratory, as well as C57BL/6J wild-type animals. NCR-NU immunodeficient mice were purchased from Taconic. Mice were housed in a pathogen-free facility at the University of Texas MD Anderson Cancer Center (MDACC). All manipulations were performed under Institutional Animal Care and Use Committee (IACUC)-approved protocols.

Human Samples: Human tissue slides containing cases of acute and chronic pancreatic inflammation were purchased from US Biomax, Inc. and used for immunofluorescence staining following the protocol described below.

### In Vivo Experiments

Induction of acute pancreatitis. Animals of 4-6 weeks were fasted for 12 hours before receiving 16 injections of caerulein (50 μg/kg) (Sigma-Aldrich) over two consecutive days (Mayerle, 2013). Control mice received injections of pyrogen-free PBS. Mice were examined daily for health conditions and sacrificed at the indicated time points by CO2 asphyxiation and cervical dislocation.

Survival studies. KRAS expression, in mice recovered from inflammation or in mice that underwent orthotopic transplantation, was induced and maintained through doxycycline administration (one injection of 4 ug/g IP), followed by feeding mice with doxycycline (2 g/l) in drinking water supplemented with sucrose (20 g/l). Mice were then monitored over time for tumor development by magnetic resonance imaging (see below).

Orthotopic transplantation. 6-9-week old female NCR-NU mice, upon anesthesia with isoflurane, were transplanted orthotopically with 2.5×10⁵ epithelial cells derived from iKRAS organoids re-suspended in modified PDEC medium and Matrigel (Corning) (1:1 ratio) (see below Organoid Culture). KRAS expression was induced soon after transplantation and mice were then monitored for tumor development by magnetic resonance imaging.

Histopathology, immunohistochemistry and immunofluorescence: Tissue specimens were fixed overnight in 4% buffered PFA, transferred to 70% ethanol and then embedded in paraffin using Leica ASP300S processor. For histopathological analysis, pancreata were sectioned (Leica RM2235) and serial slides were collected. For every series one section was stained with hematoxylin and eosin and remaining sections were kept for either immunofluorescence or immunohistochemical analysis. Histological samples were processed as previously described (Viale et al., 2014). In brief, after cutting, baking and deparaffinization, sections underwent antigen retrieval using Citra-Plus Solution (BioGenex) according to specifications. For immunohistochemistry staining, endogenous peroxidases were inactivated by 3% hydrogen peroxide and non-specific signals were blocked using 3% BSA, 10% goat serum and 0.1% Triton. Primary antibodies were applied and incubated overnight at 4° C. ImmPress HRP IgGs (Vector Lab) were used as secondary antibodies and ImmPact Nova RED (Vector Lab) was used for detection. Images were captured with a Nikon DS-Fi1 digital camera using a wide-field Nikon Eclipse-Ci microscope. For immunofluorescence staining, secondary antibodies conjugated with Alexa-488 and Alexa-555 (Molecular Probes) were used. Fluorescein labeled Dolichos Biflorus Agglutinin (DBA) (Vector Labs) was used to detect ductal cells when indicated. DAPI nuclear counterstaining was also performed. Images were captured with a Hamamatsu C11440 digital camera, using a wide-field Nikon Eclipse-Ni microscope. For organoids characterization images were acquired using a Nikon high-speed multiphoton confocal microscope A1 R MP.

The following primary antibodies were used: α-Amylase (Sigma-Aldrich), CK19 (ProteinTech), GFP (Cell Signaling), NF-kB p65 (phospho Ser536) (Abcam), Cleaved Caspase3 (Cell Signaling), Egr1 (Cell Signaling), Runx1 (Abcam), Ets1 (Abcam), CD45 (eBioscience), Ki67 (Abcam), Sox9 (Millipore), IL-6 (Abcam), Stat3 (phospho Tyr705) (Cell Signaling) and DCLK1 (Abcam).

For GFP detection in pancreatic tissue reconstituted through organoid injection, samples were fixed overnight in 4% buffered PFA at 4C, transferred to PBS+Sucrose 20% and embedded in OCT Compound (Tissue-Tek). Specimens were cryosectioned (Leica CM1950) and dried at room temperature for 10 minutes before DAPI nuclear counterstaining and image acquisition.

Image quantification: For quantification of spheroids size nine 4×-magnification fields representing organoids culture from three biological replicates each experimental group were analyzed with ImageJ expressing organoids area as pixels. Images used for quantification were captured with a Cool-SNAP ES² digital camera using a wide-field Nikon Eclipse-Ti microscope.

For ADM quantification two hematoxylin/eosin-stained 10×-magnification fields of whole-mount pancreata from two mice each experimental group were analyzed with ImageJ. ADM areas, expressed in pixels, were normalized to control (rechallenged) mice considered as reference. Images used for quantification were captured with a Nikon DS-Fi1 digital camera using a wide-field Nikon Eclipse-Ci microscope.

For Cleaved Caspase-3 (CC3) immunostaining quantification, images captured with a Hamamatsu C11440 digital camera using a wide-field Nikon Eclipse-Ni microscope were electronically processed to remove autofluorescence (e.g.: red blood cells) using Adobe Photoshop. Images, then, were analyzed with ImageJ and CC3 specific signal, expressed as area (pixels), was normalized to total pancreatic parenchyma using DAPI. From six to nine 4×-magnification fields of whole-mount pancreata from three mice each experimental group were analyzed.

For quantification of inflammation-induced TFs, automatic image segmentation using Matlab (The MathWorks, Inc.) was performed. Otsu's thresholding method and marker-controlled watershed algorithm was used to segment and specify the nuclear regions of each single cell and the mean pixel intensity of each marker in each segmented area was quantified. For sox9 staining, before quantification, cells positive for DBA staining were excluded. Violin graphs in log scale were used for data representation. An average of 3,800 nuclei from at least 7 40× fields of pancreatic tissue from 3 to 5 mice each experimental group were counted and used for the analysis.

Magnetic resonance imaging: Animals were imaged on a 4.7T Bruker Biospec (Bruker BioSpin) equipped with 6-cm inner-diameter gradients and a 35-mm inner-diameter volume coil. Multi-slice T2-weighted images were acquired in coronal and axial geometries using a rapid acquisition with relaxation enhancement (RARE) sequence with TR/TE of 2,000/38 ms, matrix size 256×192, 0.75-mm slice thickness, 0.25-mm slice gap, 4×3-cm FOV, 101-kHz bandwidth, 3 NEX. Axial scan sequences were gated to reduce respiratory motion.

### In Vitro Experiments

Organoid culture: Organoids (cystic spheroids) cultures were performed as previously described (Agbunag et al., 2006; Deramaudt et al., 2006; Schreiber et al., 2004) with some modifications using both wild-type or iKRAS animals. Briefly, pancreata from age matched control animals and animals that underwent a 4-week recovery from acute pancreatitis were harvested and kept on ice before processing. Upon mechanical disruption, tissues were washed twice with G solution (HBSS (Gibco), 5 mg/ml D-Glucose (Sigma Aldrich), 100 μg/ml Penicillin/Streptomycin (Gibco)) and incubated at 37° C. for 45 min (Collagenase IV (Gibco)-Dispase II (Roche), 2 mg/ml) for enzymatic digestion. Cells were then centrifuged and further digested with 0.25% Trypsin (Gibco) for 5 min at 37° C. to obtain a single cell suspension. Cells were then wash twice with PBS and plated on Collagen IV-coated plates (Corning) in modified PDEC medium: DMEM/F12 1:1 supplemented with 2.5 mM L-Glutamine, 15 mM HEPES Buffer (HyClone), 5 mg/ml D-Glucose (Sigma Aldrich), 1.22 mg/ml Nicotinamide (Sigma Aldrich), 5 nM 3,3,5-Tri-iodo-L-thyronine (Sigma Aldrich), 0.5 μM Hydrocortisone Solution (Sigma Aldrich), 100 ng/ml Cholera Toxin (Sigma Aldrich), 0.5% Insulin-Transferrin-Selenium+(BD), 100 μg/ml Penicillin/Streptomycin (Gibco), 0.1 mg/ml Soybean Trypsin Inhibitor (Sigma Aldrich), 20 ng/ml EGF (Peprotech), 25 μg/ml Bovine Pituitary Extract (Invitrogen), and 5% Nu Serum IV Culture Supplement (BD). After 2 days the supernatant cellular fraction was harvested and replated in PDEC-Matrigel (Corning) (1:1.5 ratio). Organoids were then passaged every 7-10 days at low confluency.

CD45+ cells isolation, organoid co-culture and Hyper-IL6 treatment. At 24 hours after the last injection of caerulein pancreata were harvested and cells isolated following the protocol described above besides that no trypsin digestion was performed in order to preserve surface antigens. After digestion pancreata were then filtered through a 45 μm nylon mesh to separate epithelial structures from other cells. After filtration CD45+ cell fraction was purified with EasySep™ Mouse Biotin Positive Selection Kit (StemCell Technologies) following the manufacturer's protocol using an anti-CD45-Bio antibody (30-F11, eBioscience). Purity (˜95-98%) of isolated cells was checked by flow cytometry using SA-APC. Isolated CD45+ cells were then suspended in modified PDEC medium and used for setting cocultures with epithelial organoids. Briefly, iKRAS epithelial cells from organoids never exposed to inflammation were plated in PDEC-Matrigel mix into high-density pore transwell (Corning, Inc.) then inserted in a 6-well plate containing the purified CD45+ cells suspended in modified PDEC media (2 ml/well). After one week of co-culture, organoids were collected and reseeded in ‘conventional’ modified PDEC-Matrigel. For Hyper-IL6 experiments organoids were plated in PDEC-Matrigel in presence of 200 ng/ml of Hyper-IL6 for 24 hours. Hyper-IL6 was kindly provided by Dr. Stefan Rose-John.

Flow Cytometry and Single-Cell Sorting: For flow-cytometry, sample acquisition was carried out using a BD FACS Canto II or LS-Fortessa cytometers (BD Biosciences) at the MD Anderson South Campus Flow Cytometry and Cell Sorting Facility. Data were analyzed by BD FACSDiva or FlowJo (Tree Star) excluding doublets and dead cells (DAPI positive) at the time of the gating-strategy. For purity assessment of isolated inflammatory cells, digested pancreata labelled with anti CD45-Bio (eBioscience) antibody were stained with SA-APC (eBioscience) before and after EasySep purification.

For cell sorting, single cell suspensions were obtained from pancreata of DCLK1-DTR-zsGreen mice and wild type control animals following the mechanical disruption and enzymatic digestion described above. After adding 1 g/ml DAPI (Thermo Fisher) to exclude dead cells samples were processed with a BD FACS Influx cell sorter (BD Bioscience) using cells isolated from wild-type animals to set the sorting gates. Both zsGreen-positive and negative fractions of cells from DCLK1-DTR-zsGreen digested pancreata were collected and used for establishing organoid cultures.

Cytokine detection: Media conditioned by CD45+ cells isolated from Caerulein-treated pancreata were collected at indicated time points and analyzed by Mouse Inflammatory Cytokines Multi-Analyte ELISArray Kit (Qiagen). Measurements were repeated multiple times from independent wells according to the manufacturer protocols. Absorbance was read by PHERAStar HTS microplate reader (BMG Labtech).

Serum Amylase and LDH detection: Blood was drawn from retro orbital vein at 24 hours from the first injection of Caerulein (after 8 injections) and collected in Z-Serum Separator Clot Activator tubes (Greiner Bio-One). After 30 minutes at room temperature samples were centrifuged for 10 min to separate the clot from the serum, samples then were aliquoted and stored at −80 C. The concentration of pancreatic amylases and lactate dehydrogenase in the serum was measured using respectively the Amylase Assay Kit (Abcam) and Mouse LDH/Lactate Dehydrogenase ELISA Kit (LifeSpan Biosciences) according to specification.

Immunoblotting: After washing in ice-cold PBS, collected cells were pelleted and resuspended in RIPA buffer with proteinase and phosphatase inhibitors. Lysates were then centrifuged at 14,000 rpm for 20 min at 4° C. to eliminate debrides. Protein concentrations were assessed using the DC Protein Assay Kit (Biorad). Samples were loaded on a 5-15% gradient Mini-Protean TGX Precast Gels for the SDS-PAGE and then transferred onto PVDF membranes according to standard protocols. Membranes were incubated with indicated primary antibodies, washed, and probed with HRP-conjugated secondary antibodies. Protein specific signals were identified by chemiluminescence upon film exposure. The following antibodies were used: Stat3 (phospho Tyr705) (Cell Signaling), Stat3 (Cell Signaling), Egr1 (Cell Signaling), Runx1 (Abcam), Ets1 (Abcam), Sox9 (Millipore) and Vinculin (Sigma-Aldrich)

CyTOF immunophenotyping: Metal-labeled antibodies against cell surface markers were purchased from DVS Sciences. A single cell suspension was obtained as described above (CD45+ cells isolation section) from pancreatic tissue undergone Caerulein-induced inflammation and harvested after 24 hours from the last Caerulein injection. The cells were depleted of erythrocytes by hypotonic lysis. After washing the samples were centrifuged and resuspended in a PBS+0.5% BSA solution with a mix of all surface antibodies and incubated at 4 C for 1 hour. Cells were then washed once and incubate with 25 uM Cisplatin for 1 min for the viability staining. The fixation and permeabilization step was carried out using Fixation/Permeabilization Solution kit (BD Biosciences) for 20 minutes. After washing the step of intracellular staining was performed incubating the cells in a PBS+0.5% BSA solution with the IL6_167Er antibody (FluidiGM) for 1 hour. After washing samples were incubated with MAXPAR®Nucleic Acid Intercalator-Ir (DVS Sciences) at 4° C. overnight to stain the nuclei and analyzed with CyTOF instrument (DVS Sciences) in the Flow Cytometry and Cellular Imaging Core Facility at M.D. Anderson Cancer Center. Data were processed with FlowJo (Tree Star) and viSNE. The following markers were used to define different immune populations: CD45_89Y, CD68 145Nd, CD11b 148Nd, F4/80_173Yb, CD4_115In, CD8a_168Er, B220_176Yb, NK1.1_170Er.

RNA-Seq Data Analysis: Total RNA was extracted from C57BL6 WT organoids using the RNeasy Mini Kit (Qiagen) following manufacturer instructions and analyzed using the RNA Nano kit on the Agilent Bioanalyzer (Agilent Technologies). Paired-end multiplex sequencing of samples was performed on the Illumina HiSeq 2000 sequencing platform. After quality filtering according to the Illumina pipeline, 76 bp paired-end reads were aligned to the mm10 mouse reference genome and to the Mus musculus transcriptome (GRCm38) using TopHat (version 2.1.0) (Kim et al., 2013) with options “-r 148 --no-mixed --no-discordant”. At the gene level, expression counts were estimated using featureCounts (Rsubread version 1.5.1) (Liao et al., 2014), summing reads across all exons as annotated in NCBI GRCm38/mm10, with option “--largestOverlap”. Both coding and long noncoding genes were retained for downstream analyses.

Normalization and differentially expressed genes in two biological replicates of control (Ctrl) and in three treatment replicates (Post-CAE) were identified using EdgeR R-package (version 3.2.2) (Robinson et al., 2010). Prior to normalization, genes with low expression (less than 0.5 CPM, Count Per Million, in the two Ctrl samples or in the three Post-CAE samples) were removed from further analyses. Normalization factors were computed on the filtered data matrix using the Trimmed Mean of M (TMM) method, followed by voom mean-variance transformation in preparation for Limma linear modeling (Law et al., 2014). A linear model was fitted to each gene, and empirical Bayes moderated t-statistics were used to assess differences in expression (Smyth, 2004). The expression levels were calculated by using the fragments per kilobase per million reads method (FPKM). Genes were identified as differentially expressed (DEGs) when the following criteria were met: log 2 of the fold-change (FC);≥0.8; false discovery rate (FDR)<0.05; at least 1 FPKM in all samples in one or both conditions. DEGs were hierarchically clustered using pheatmap R package (Kolde R: pheatmap: Pretty Heatmaps 2015) utilized a Euclidean distance metric and complete linkage rule, after setting the minimum FPKM value to 0.1 and after log 2-transformation.

For the Gene Set Enrichment Analysis (GSEA) (Subramanian et al., 2005) all genes were ranked by signed P-value and 1000 gene set permutations were performed to assess the statistical significance. Gene sets with FDR<0.05 were considered significant. Gene sets background was built using the hallmark gene signature, downloaded from The Molecular Signatures Database (MSigDB). An additional gene set named ‘Development/Progression’ was obtained from Reichert et al. (Reichert et al., 2013) considering 310 genes commonly up-regulated in epithelial pancreatic mouse cells during development and KRAS-G12D dependent carcinogenesis (tumor progression). Ingenuity Pathway Analysis (IPA, Ingenuity® Systems; Redwood City, Calif.) was carried out using a commercial software.

ChIP-seq Data Analysis: Short reads obtained from Illumina HiSeq 2000 were quality filtered according to the Illumina pipeline. Reads were then mapped to the human mm10 reference genome using Bowtie2 v2.2.6 (54) with the “-very-sensitive” preset of parameters. Reads that did not align to the nuclear genome or aligned to the mitochondrial genome were removed. Moreover, duplicate reads were marked and removed using SAMtools (55). Peak calling vs. the input genomic DNA was performed using MACS 1.4 (Zhang et al., 2008) using the “gsize mm”, “--nomodel” and “--shiftsize 125” flags and arguments. A matched input was used as control. Peaks with a P-value>1E-10, both ChIP vs. input DNA and ChIP vs. ChIP, and those blacklisted by the ENCODE consortium analysis of artifactual signals in mouse genome were removed using bedtools (Quinlan and Hall, 2010). To classify acetylated regions based on their genomic location and to assign them to the nearest transcription start site (TSS), the September 2017 RefSeq annotation of the mm10 version of the mouse genome was given as input to the annotatePeaks script from HOMER package (Heinz et al., 2010) Each peak was classified as either TSS-proximal or TSS-distal, depending on its distance (< or >2.5 kb, respectively) from TSS.
1. Motif enrichment analysis
2. In order to identify statistically overrepresented motifs corresponding to known TF binding sites, a collection of position-specific weight matrices (PWMs) was obtained as described in Diafera & Balestrieri et al. (Diaferia et al., 2016). Significantly overrepresented PWMs were identified using a modified version of Pscan, in which a t-test was implemented in place of the original z-test (Zambelli et al., 2009). Any PWM showing a P-value equal or lower than 1E-5 was considered as significantly overrepresented.

To identify PWMs enriched in the promoter regions of up-regulated genes, a window of 600 bp (−500 and +100 bp relative to TSS) was considered and a set including all Refseq gene promoters was used as background. For the set of differential acetylated regions, the acetylated enhancers were compared with the set of mouse enhancers in the FANTOM5 collection (Andersson et al., 2014) using a window of ±250 bp around enhancer centers.

Statistical analysis: In vitro and in vivo data are presented as the mean±standard deviation. Statistical analyses were calculated using a two-tailed Student's t-test after the evaluation of variance. For survival studies mice were randomized to the experimental groups and results analyzed with a log-rank (Mantel-Cox) test and expressed as Kaplan-Meier survival curves.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Agbunag, Methods Enzymol 407, 703-710 (2006).
Andersson et al., Nature 507, 455-461 (2014).
Asangani et al., Nature 510, 278-282 (2014).
Bowie, Handbook of Biologically Active Peptides_Caeruleins. (Elsevier Inc, ed. Second Edition, 2013).
Carnahan et al., Mol Cancer Ther 9, 2399-2410 (2010).
Delmore et al., Cell 146, 904-917 (2011).
Deramaudt et al., Molecular and cellular biology 26, 4185-4200 (2006).
Diaferia et al., The EMBO journal 35, 595-617 (2016).
Dittmer, Seminars in cancer biology 35, 20-38 (2015).
Falchook et al., Clinical cancer research: an official journal of the American Association for Cancer Research, (2019).
Filippakopoulos et al., Nature 468, 1067-1073 (2010).
Fukuda et al., Cancer cell 19, 441-455 (2011).
Genovese et al., Nature 542, 362-366 (2017).
Gidekel Friedlander et al., Cancer cell 16, 379-389 (2009).
Grivennikov et al., Cancer cell 15, 103-113 (2009).
Grivennikov et al., Cell 140, 883-899 (2010).
Guerra et al., Cancer cell 19, 728-739 (2011).
Halbrook et al., Cell Mol Gastroenterol Hepatol 3, 99-118 (2017).
Hayden, Genes & development 26, 203-234 (2012).
Heinz et al., Mol Cell 38, 576-589 (2010).
Houbracken et al., Gastroenterology 141, 731-741, 741 e731-734 (2011).
International Patent Publication No. WO 00/37504
International Patent Publication No. WO 01/14424
International Patent Publication No. WO 98/42752
International Patent Publication No. WO1995001994
International Patent Publication No. WO1998042752
International Patent Publication No. WO2008/121949
International Patent Publication No. WO2009/101611
International Patent Publication No. WO2009/114335
International Patent Publication No. WO2010/027827
International Patent Publication No. WO2011/053435
International Patent Publication No. WO2011/066342
International Patent Publication No. WO2014/087413
Joseph et al., Proceedings of the National Academy of Sciences of the United States of America 107, 14903-14908 (2010).
Kapoor et al., Cell 158, 185-197 (2014).
Karin and Clevers, Nature 529, 307-315 (2016).
Kim et al., Genome Biol 14, R36 (2013).
Kopp et al., Cancer cell 22, 737-750 (2012).
Langmead and Salzberg, Nature methods 9, 357-359 (2012).
Law et al., Genome Biol 15, R29 (2014).
Lesina et al., Cancer cell 19, 456-469 (2011).
Li et al., Bioinformatics 25, 2078-2079 (2009).
Liao et al., Bioinformatics 30, 923-930 (2014).
Liou et al., The Journal of cell biology 202, 563-577 (2013).
Mantovani et al., Nature 454, 436-444 (2008).
Mayerle, in Pancreapedia: Exocrine Pancreas Knowledge Base. (Pancreapedia, 2013).
Mazur et al., Nature medicine 21, 1163-1171 (2015).
Miyatsuka et al., Genes & development 20, 1435-1440 (2006).
Notta et al., Nature 538, 378-382 (2016).
Prevot et al., Gut 61, 1723-1732 (2012).
Quinlan and Hall, Bioinformatics 26, 841-842 (2010).
Real, Gastroenterology 124, 1958-1964 (2003).
Reichert et al., Genes & development 27, 288-300 (2013).
Robinson et al., Bioinformatics 26, 139-140 (2010).
Sandgren et al., Cell 61, 1121-1135 (1990).
Scheitz, The EMBO journal 31, 4124-4139 (2012).
Schreiber et al., Gastroenterology 127, 250-260 (2004).
Shi et al., Oncogene 32, 1950-1958 (2013).
Smyth, Stat Appl Genet Mol Biol 3, Article 3 (2004).
Steele et al., British journal of cancer 108, 997-1003 (2013).
Storz, Nat Rev Gastroenterol Hepatol 14, 296-304 (2017).
Subramanian et al., Proceedings of the National Academy of Sciences of the United States of America 102, 15545-15550 (2005).
Thiel et al., J Cell Physiol 193, 287-292 (2002).
Thompson (ed.), The Cytokine Handbook, Academic Press, San Diego, 1994.
U.S. Pat. No. 5,844,905
U.S. Pat. No. 5,885,796
U.S. Pat. No. 6,207,156
U.S. Pat. No. 6,232,287
U.S. Pat. No. 8,017,114
U.S. Pat. No. 8,119,129
U.S. Pat. No. 8,329,867
U.S. Pat. No. 6,528,481
U.S. Pat. No. 7,452,964
U.S. Pat. No. 7,671,010
U.S. Pat. No. 7,781,565
U.S. Pat. No. 8,450,278
U.S. Pat. No. 8,507,445
U.S. Patent Publication No. 2006/0034925
U.S. Patent Publication No. 2006/0115537
U.S. Patent Publication No. 2006/0223114
U.S. Patent Publication No. 2006/0234299
U.S. Patent Publication No. 2007/0148095
U.S. Patent Publication No. 2012/0141550
U.S. Patent Publication No. 2013/0138032
U.S. Patent Publication No. 2014/0024610
Viale et al., Nature 514, 628-632 (2014).
Virchow, J. B. Lippincott, Philadelphia, (1863).
Westphalen et al., Cell stem cell 18, 441-455 (2016).
Ying et al., Cell 149, 656-670 (2012).
Ying et al., Genes & development 30, 355-385 (2016).
Yu et al., Nature reviews. Cancer 9, 798-809 (2009).
Zambelli et al., Nucleic acids research 37, W247-252 (2009).
Zhang et al., Cancer research 73, 6359-6374 (2013).
Zhang et al., Genome Biol 9, R137 (2008).

What is claimed is:

1. A method of treating pancreatitis and/or preventing pancreatic cancer in a subject comprising administering an effective amount of an acinar-to-ductal metaplasia (ADM) inducer to the subject.

2. The method of claim 1, wherein the method comprises treating or preventing pancreatitis in the subject.

3. The method of claim 1 or 2, wherein the method comprises preventing pancreatic cancer in the subject.

4. The method of any of claims 1-3, wherein the pancreatic cancer is pancreatic ductal adenocarcinoma (PDAC).

5. The method of claim 1 or 2, wherein the pancreatitis is chronic pancreatitis.

6. The method of claim 1 or 2, wherein the pancreatitis is acute pancreatitis.

7. The method of any of claims 1-6, wherein the ADM inducer is an epigenetic modifier.

8. The method of claim 7, wherein the epigenetic modifier is a Bromodomain extra-terminal motif (BET) inhibitor.

9. The method of claim 8, wherein the BET inhibitor is a BRD2 inhibitor, BRD3 inhibitor, BRD4 inhibitor, or BRDT inhibitor.

10. The method of claim 8, wherein the BET inhibitor is a BRD4 inhibitor.

11. The method of claim 10, wherein the BRD4 inhibitor is INCB054329, GSK525762A/I-BET762, INCB054329, ABBV-075, OTX015/MK-8628, GSK2820151/I-BET151, PLX51107, ABBV-744, or AZD5153.

12. The method of any of claims 1-11, wherein the ADM inducer is a mitogen-activated protein kinase (MAPK) agonist.

13. The method of any of claim 12, wherein the MAPK agonist is a BRAF inhibitor, TGFα, or EGF.

14. The method of claim 12 or 13, wherein the MAPK agonist is TGFα or EGF.

15. The method of claim 12 or 13, wherein the MAPK agonist is a BRAF inhibitor.

16. The method of claim 15, wherein the BRAF inhibitor is PLX4032 (Vemurafenib), GDC-0879, PLX-4720, sorafenib, dabrafenib (GSK2118436), AZ 628, LGX818, or NVP-BHG712.

17. The method of claim 15 or 16, wherein the BRAF inhibitor is an SOS activator and/or GEF inhibitor.

18. The method of claim 15 or 16, wherein the BRAF inhibitor is vemurafenib.

19. The method of any of claims 1-18, wherein the subject is determined to be RAF wild-type.

20. The method of any of claims 1-19, wherein the subject is not administered a MEK inhibitor.

21. The method of claim 20, wherein the MEK inhibitor is trametinib.

22. The method of any of claims 1-21, wherein administering the ADM inducer prevents development of KRAS mutations in the subject.

23. The method of any of claims 1-22, wherein administering the ADM inducer prevents or decreases tissue damage and/or inflammation in pancreatic cells as compared to a subject not administered an ADM inducer.

24. The method of claim 23, wherein decreased inflammation is measured by decreased inflammatory infiltration, serum inflammatory biochemical markers, edema, or pain.

25. The method of claim 23 or 24, wherein decreased tissue damage is measured by lipase, amylase, trypsinogen, and/or lactate dehydrogenase.

26. The method of any of claims 1-25, wherein the subject is human.

27. The method of any of claims 1-26, further comprising administering at least a second therapy.

28. The method of claim 27, wherein the second therapy is an anti-inflammatory agent, an immunotherapy, and/or supportive care.

29. The method of claim 27 or 28, wherein the second therapy is administered concurrently with the ADM inducer.

30. The method of any of claims 27-29, wherein the second therapy is administered sequentially with the ADM inducer.

31. The method of any of claims 27-30, wherein the second therapy is an anti-inflammatory agent.

32. The method of claim 31, wherein the anti-inflammatory agent is a non-steroidal anti-inflammatory drug (NSAID) or a steroid.

33. The method of any of claims 1-32, wherein the ADM inducer is administered orally, intraadiposally, intradermally, intramuscularly, intranasally, intraperitoneally, intrarectally, intravenously, liposomally, locally, mucosally, parenterally, rectally, subcutaneously, sublingually, transbuccally, transdermally, via a catheter, via a lavage, via continuous infusion, via infusion, via inhalation, via injection, or via local delivery.

34. The method of any of claims 1-33, wherein the ADM inducer is administered once to the subject.

35. The method of any of claims 1-34, wherein the ADM inducer is administered two or more times to the subject.

36. A composition comprising an effective amount of an ADM inducer for use in the treatment of pancreatitis and/or prevention of pancreatic cancer in a subject.

37. The composition of claim 36, wherein the ADM inducer is a MAPK agonist.

38. The composition of claim 37, wherein the MAPK agonist is a BRAF inhibitor, TGFα, or EGF.

39. The composition of claim 38, wherein the MAPK agonist is a BRAF inhibitor.

40. The composition of claim 39, wherein the BRAF inhibitor is PLX4032 (Vemurafenib), GDC-0879, PLX-4720, sorafenib, dabrafenib (GSK2118436), AZ 628, LGX818, or NVP-BHG712.

41. The composition of claim 39 or 40, wherein the BRAF inhibitor is vemurafenib.

42. The composition of claim 36, wherein the ADM inducer is an epigenetic modifier.

43. The composition of claim 42, wherein the epigenetic modifier is a Bromodomain extra-terminal motif (BET) inhibitor.

44. The composition of claim 43, wherein the BET inhibitor is a BRD4 inhibitor.

45. The composition of claim 44, wherein the BRD4 inhibitor is INCB054329, GSK525762A/I-BET762, INCB054329, ABBV-075, OTX015/MK-8628, GSK2820151/I-BET151, PLX51107, ABBV-744, or AZD5153.

46. The method of claim 42, wherein the epigenetic modifier is a small molecule, peptide, siRNA, sgRNA, PROTAC or degron.

47. The composition of any of claims 36-46, wherein the subject is human.

48. The composition of any of claims 36-47, wherein the pancreatitis is chronic pancreatitis.

49. The composition of any of claims 36-48, wherein the pancreatitis is acute pancreatitis.

50. The composition of any of claims 36-49, wherein the pancreatic cancer is PDAC.

51. The composition of any of claims 36-50, wherein the ADM inducer prevents development of KRAS mutations, tissue damage, and/or inflammation in the subject.

52. The composition of any of claims 36-51, further comprising at least a second therapy.

53. The composition of claim 52, wherein the second therapy is an anti-inflammatory agent and/or immunotherapy.

54. The composition of claim 52 or 53, wherein the second therapy is an anti-inflammatory agent.

55. The composition of claim 54, wherein the anti-inflammatory agent is a steroid or an NSAID.

56. A method of inhibiting pancreatic tissue damage and/or inflammation in a subject comprising administering an effective amount of an ADM inducer to the subject.

57. The method of claim 56, wherein the ADM inducer is a MAPK agonist.

58. The method of claim 57, wherein the MAPK agonist is a BRAF inhibitor, TGFα, or EGF.

59. The method of claim 58, wherein the MAPK agonist is a BRAF inhibitor.

60. The method of claim 59, wherein the BRAF inhibitor is vemurafenib.

61. The method of any of claims 56-60, wherein the ADM inducer is an epigenetic modifier.

62. The method of claim 61, wherein the epigenetic modifier is a Bromodomain extra-terminal motif (BET) inhibitor.

63. The method of claim 62, wherein the BET inhibitor is a BRD4 inhibitor.

64. The method of claim 63, wherein the BRD4 inhibitor is INCB054329, GSK525762A/I-BET762, INCB054329, ABBV-075, OTX015/MK-8628, GSK2820151/I-BET151, PLX51107, ABBV-744, or AZD5153.

65. The method of claim 61, wherein the epigenetic modifier is a small molecule, peptide, siRNA, sgRNA, PROTAC or degron.

66. A method of treating pancreatitis in a subject comprising administering an effective amount of an ADM inducer to the subject, wherein the ADM inducer is a MAPK agonist or an epigenetic modifier.

67. The method of claim 66, wherein the ADM inducer is a MAPK agonist, wherein the MAPK agonist is a BRAF inhibitor.

68. The method of claim 67, wherein the BRAF inhibitor is vemurafenib.

69. The method of claim 66, wherein the ADM inducer is an epigenetic modifier, wherein the epigenetic modifier is a BRD4 inhibitor.

70. The method of claim 69, wherein the BRD4 inhibitor is INCB054329, GSK525762A/I-BET762, INCB054329, ABBV-075, OTX015/MK-8628, GSK2820151/I-BET151, PLX51107, ABBV-744, or AZD5153.
